(19) United States
(12) Patent Application Publication
(10) Pub. No.: US 2002/0090381 A1
(43) Pub. Date: Jul. 11, 2002

(54) SYSTEM FOR CONTROLLING IMMUNE SYSTEM RESPONSE TO ANTIGEN

(76) Inventors: H. KIM BOTTOMLY, NEW HAVEN, CT (US); MICHAEL J. CAPLAN, WOODBRIDGE, CT (US); HOWARD B. SOSIN, FAIRFIELD, CT (US)

Correspondence Address: BRENDA HERSCHBACH JARRELL, CHOATE HALL & STEWART, EXCHANGE PLACE, 53 STATE, BOSTON, MA, 021092891

(21) Appl. No.: 09/290,029

(22) Filed: Apr. 9, 1999

(51) Int. Cl. A61K 39/395; C12N 5/16; C07K 17/00; C07K 14/00

(52) U.S. Cl. 424/198.1; 435/326; 530/351

(57) ABSTRACT

The present invention provides compositions and methods for regulating immune system reactions by biasing T cell responses away from Th1 or Th2 responses in a pre-determined manner. Control is effected at the stage of antigen/APC encounter and/or at the stage of APC/T cell encounter. In preferred embodiments, a Th1 or Th2 response is inhibited through induction of the alternative response. The inventive methods and reagents are particularly useful for the management of autoimmune disorders, allergy, and asthma.

### BACKGROUND OF THE INVENTION

In general, an adaptive immune system response to an antigen is thought to have two components: a “cell-mediated” response, carried out by T-cells, and “humoral” response, mediated by antibodies that are produced by B cells. The combination of these two responses allows the detection and elimination both of extracellular antigens, which are often recognized by antibodies, and of antigens that are found inside cells.

B and T cells have distinct receptors specialized for the recognition of antigens. The B cell receptor is an immunoglobulin molecule that is positioned on the B cell surface and recognizes a particular exposed antigen epitope. When this receptor binds an antigen, the B cell becomes activated to multiply and to secrete a soluble version of the receptor immunoglobulin. This soluble molecule will bind and clear the antigen. The T cell receptor also recognizes a particular antigen epitope, but only if it is presented as a processed form of the antigen, displayed in the context of a major histocompatibility (MHC) molecule on the surface of an antigen presenting cell (APC). Any cell that captures and processes antigen so that antigen fragments are displayed in an MHC molecule on its surface can act as an APC.

The relationship between humoral and cell-mediated responses is complex. Initially, all B cells express a class of antibody known as “IgM”. In fact, the B cell receptor itself is an IgM antibody. However, when the B cell encounters a helper T cell (described below) that has been activated by the same antigen, a “class switch” may be induced in the B cell, so that the B cell begins to secrete a different class of antibody. The type of helper T cell that the B cell encounters determines the class of antibody that the B cell is induced to express. Because antibodies of different classes have different activities, the presence of antigen-activated helper T cells (i.e., of a cell-mediated response) in the vicinity of activated B cells can alter the nature of the humoral response to the antigen. T cells present during a B cell response can also affect the maturation of that response. That is, when a B cell is stimulated to divide as a result of its activation by antigen and helper T cells, certain structural rearrangements and sequence alterations occur in the portions of its genome that encode immunoglobulin. As a result of these changes, progeny of the original B cell may express immnunoglobulins not only of a different class from that expressed by the parent cell, but also of varied affinity for the antigen. Re-exposure of such progeny B cells to antigen results in the selective amplification of B cells that produce high-affinity antibodies to the antigen.

Initiation of a cell-mediated response is somewhat more complex. There are different classes of T cells. In particular, T cells are divided into two major subsets based both on receptor specificity and on cell surface markers. “CD4” T cells are so named because they express CD4 glycoproteins on their surfaces; they also recognize peptide antigen fragments presented in the context of class II MHC molecules. “CD8” T cells express CD8 glycoproteins on their surfaces and recognize peptide antigens presented in the context of class I MHC molecules. In general, the two classes of MHC molecules differ in the sources of peptide antigen that they display. Class I MHC molecules present antigen fragments generated by cytosolic degradation of antigen; class II MHC molecules present fragments generated by degradation of antigen in intracellular vesicles. Most cells express MHC class I molecules on their surfaces, but only specialized cells, including dendritic cells, macrophages, and B cells, express both MHC class I and MHC class II molecules. Such cells are referred to as “professional” APCs (pAPCs).

Exposure of a naive CD4- or CD8-expressing T cell to a presented antigen is required for that cell to recognize antigen, but is not sufficient to fully activate the T cell and induce its proliferation, as is required to achieve efficient T cell immunity. In particular, a T cell will not become fully activated by exposure to an antigen unless additional or co-stimulatory signals are also delivered by pAPCs. Thus, pAPCs are required for T cell activation. Moreover, activation of naive CD8 T cells may require the presence of CD4 T cells that have already been activated against the same antigen in order to provide certain required co-stimulatory signals. Once a T cell is activated, or “primed” to a particular antigen, it can be stimulated by any APC that presents an appropriate T cell receptor ligand; pAPCs are not necessarily required.

An activated T cell synthesizes certain “effector” molecules and is referred to as an effector cell. There are two general categories of effector T cells, defined on the basis of the effector proteins they release and therefore the biological events that they mediate. Activated CD8 T cells are referred to as “killer” or “cytolytic” T cells (CTL) because of their ability to kill target cells that display (in the context of an MHC class I molecule) the antigen against which they have been activated. Activated CD4 T cells are referred to as “helper” cells because of the many roles they play in supporting immune reactions mediated by other cells and immune agents. In particular, once a helper cell has been activated to a particular antigen, a subsequent encounter with the antigen in the context of an MHC class II molecule stimulates the cell to produce effector molecules called cytokines, that regulate the activity of target cells expressing cytokine receptors. Different subtypes of activated helper cells produce different arrays of cytokines, and therefore have different effects on target cells.

There are two main categories of helper cell subtypes: Th1 and Th2. A particular naive CD4 T cell differentiates into a cell of one or the other subtype as part of its activation process; which subtype it selects is determined by which cytokines are present in the environment of the T cell during its activation. In particular, a Th1 response is selectively induced when activated T cells encounter antigen in the presence of IL-12, IFNα, and/or IFNγ; a Th2 response is induced when activated T cells encounter antigen in the presence of IL-4.

Once a CD4 T cell has been activated as a Th1 or Th2 effector cell, its subsequent exposure to antigen induces it to express cytokines characteristic of a Th1 or Th2 response, respectively. For example, Th1 cells secrete the cytokines interferon γ (IFNγ) and tumor necrosis factor β (TNFβ), but do not secrete interleukin-4 (IL-4) or interleukin-5 (IL-5); Th2 cells secrete IL-4 and IL-S but not IFNγ nor TNFβ. The result of this differential cytokine secretion is that a Th1 response induces the activation of macrophages harboring infectious agents and of B cells secreting certain opsonizing antibodies, whereas a Th2 response stimulates B cells secreting primarily IgG, IgA, and IgE antibodies. A Th1 response is required for the clearance of pathogens that live and replicate in intracellular vesicles; a Th2 response provides protection against large parasites. Th2 responses are also responsible for inducing IgE-mediated allergic or asthmatic reactions, including anaphylaxis.

Th1 and Th2 responses may be mutually inhibitory. That is, when a particular antigen is presented to a collection of activated T cells, either Th1 or Th2 cells, but not both, are stimulated. Furthermore, the resulting Th1 or Th2 response inhibits the alternative response. Thus, when antigen is encountered repeatedly, it is likely that the same type of response will always be mounted for that antigen.

As is well known, an effective immune response is a body's most powerful weapon against invading pathogens. However, immune reactions can also cause devastating damage if inappropriately initiated or maintained. Autoimmune diseases, for example, represent problematic instances in which an individual's immune system inappropriately mounts a response against a self antigen.

Also, allergic and many asthmatic reactions represent undesirably extreme and/or prolonged reactions to innocuous antigens. In particular, allergic disease results when an individual exposed to a particular innocuous antigen mounts a Th2 response to that antigen. The Th2-type helper T cells activate antigen-specific B cells and also secrete IL-4, which favors IgE production by those activated B cells. Antigen-specific IgE molecules thereby produced circulate throughout the body and bind to receptors on the surfaces of mast cells, basophils, and other related cells, so that these cells become sensitized to the particular antigen. When antigen subsequently comes in contact with the mast cells, it is recognized by the surface-bound IgE molecules, which become cross-linked to one another through their interaction with antigen. This cross-linking triggers a cascade of events within the mast cells, resulting in mast cell degranulation and the release of mediators that lead to immediate (within seconds to minutes) hypersensitivity allergic reactions. In the most dramatic cases, an overzealous IgE response can result in severe anaphylaxis and even death.

One particularly problematic aspect of such an IgE response is that, once the immune response has been initiated, the IgE remains bound to the mast cells for an extended period of time. Subsequent exposures to antigen result in immediate mast cell-mediated reactions, without the requirement of upstream immune system events (e.g., differentiation and/or stimulation of Th2-type T cells). It therefore has been difficult to undue acquired hypersensitivity to a particular antigen.

There is a need for the development of technology that will reduce the danger that can be associated with immune system reactions, particularly autoimmune and/or allergic or asthmatic reactions.

### SUMMARY OF THE INVENTION

The present invention encompasses the recognition that significant control over immune system reactions can desirably be achieved through regulation of the context in which an antigen is presented to the immune system. The invention provides methods and reagents for achieving such regulation, as well as descriptions of particularly desirable applications for such techniques.

In general, the present invention provides methods and reagents for reducing the likelihood that an individual will respond to antigen exposure by mounting an undesirable Th1 or Th2 response. For example, the invention provides approaches for minimizing Th2 responses to antigens in individuals who are allergic to those antigens. The invention also provides approaches for minimized Th1 responses to autoantigens in individuals who suffer from or are susceptible to autoimmune diseases.

In one aspect, the present invention provides a method of modulating an immune system response to an antigen by (i) identifying an individual who has been or will be exposed to an antigen; and (ii) administering to the individual, concurrently with exposure to the antigen, a composition comprising at least one factor selected from the group consisting of cytokines and inducing agents, which factor is selected to bias the individual's immune response to the antigen away from a Th1 or Th2 response in a predetermined manner.

In another aspect, the invention provides a method of modulating an immune system response to an antigen by (i) isolating from an individual one or more pAPC selected from the group consisting of mature pAPC, immature pAPC, and precursors to PAPC; and (ii) exposing the isolated cells to an antigen so that mature pAPC displaying the antigen are generated, and a pre-determined set of cytokines is expressed.

In yet another aspect, the invention provides a method of modulating an immune system response to an antigen by (i) isolating from an individual one or more APC selected from the group consisting of mature pAPC, immature pAPC, and precursors to PAPC; (ii) exposing the isolated cells to an antigen so that mature pAPC displaying the antigen are generated; and (iii)contacting the antigen-exposed pAPC with T cells so that a predetermined T-cell response is inhibited.

The invention also provides a method of treating allergy by (i) identifying an individual who is allergic to an antigen; (ii) providing a composition of pAPC displaying the antigen; and (iii) contacting the composition with T cells of the individual under conditions that inhibit a Th2 response to the antigen; and a method of treating an autoimmune disorder by (i) identifying an individual who is susceptible to or has mounted an undesirable immune response against an antigen; (ii) providing a composition of pAPC displaying the antigen; and (iii) contacting the composition with T cells of the individual under conditions that inhibit a Th1 response to the antigen.

The invention further provides compositions such as, for example, a composition comprising (i) an antigen; and (ii) at least one factor selected from the group consisting of cytokines and inducing agents; and a composition comprising (i) one or more pAPC displaying an antigen and expressing a predetermined collection of cytokines, selected from the group consisting of Th1 stimulating cytokines and Th2 stimulating cytokines; and (ii) at least one factor selected from the group consisting of cytokines and inducing agents. The invention also provides a composition comprising (i) a gene encoding an antigen; and (ii) a gene encoding at least one factor selected from the group consisting of cytokines and inducing agents.

### DEFINITIONS

As used herein, the following terms have the following definitions:

“Allergic individual”: “Allergic individual” refers to an individual with sensitivities to particular antigens or allergens as exhibited by the production of IgE sufficient to cause a measurable clinical response. Such an individual has a reaction to a relatively innocuous antigen, causing a harmful immune response and/or tissue damage. Symptoms of allergy may consist of exaggerated or pathological reaction (e.g., sneezing, respiratory distress, itching, or skin rashes) to substances, situations or physical states that are without comparable effect on the average individual.

“Antigen”: means (i) any compound that elicits an immune response; and/or (ii) any compound that binds to a T cell receptor or to an antibody produced by a B-cell. Furthermore, for the purposes of the present invention the following subsets of antigens are specifically defined: an “allergen” is an antigen that (i) elicits an IgE response in an individual; and/or (ii) elicits an asthmatic reaction (whether or not such a reaction includes a detectable IgE response).

“Antigen presenting cell”: an APC is any cell that is capable of presenting antigen in a manner sufficient to induce an immune response in a naive cell or to stimulate an immune response in a previously primed cell. A “professional” APC (pAPC) is an APC that displays antigen in the context of an MHC molecule and (i) is capable of providing co-stimulatory signals and initiating a primary immune response (i.e., activating or priming a naive T cell); and/or (ii) expresses cytokines sufficient to induce an immune response in a committed T cell. Such pAPC include macrophages, dendritic cells and B cells.

“Asthmatic individual”: refers to an individual who experiences asthmatic symptoms (e.g., chronic airway inflammation characterized by eosinophilia, airway hyperresponsiveness, and excess mucus production) upon inhalation of a particular substance or antigen. Asthmatic individuals, in contrast to allergic individuals, do not necessarily exhibit a detectable production of IgE.

“Biological activity”: in respect to a peptide or linear antigen means the ability stimulate a primary and/or secondary T cell response.

“Cytokine”: A “cytokine” is a small molecule that is released from or expressed by a cell and can alter the behavior or regulate the activity of one or more immunologically relevant target cells expressing a receptor for the cytokine. Cytokines that, if expressed by a pAPC or other cell during presentation of antigen to a T cell, would induce a particular response in that T cell can be classified according to the type of response they induce in the T cell. For example, cytokines that induce a Th1 response (e.g., IL-12, IL-2, IL-18, IL-1β or fragments thereof, IFNα, and/or IFNγ, etc.) are referred to herein as “Th1 stimulating cytokines”; cytokines such that induce a Th2 response (e.g., IL-4, etc.) are referred to herein as “Th2 stimulating cytokines”. Cytokines that are produced during a Th1 response (e.g., IFNγ, TNFβ, etc.) are referred to as “Th1 cytokines”; cytokines that are produced during a Th2 response (e.g., IL-4, IL-5, etc.) are referred to as “Th2 cytokines”.

“Immature dendritic cells”: are cells that have the capacity to differentiate into mature dendritic cells. These include both proliferating and nonproliferating precursors of mature dendritic cells. For the purposes of the present invention, any cells that can be differentiated into dendritic cells in vitro (e.g., by exposure to cytokines such as GM-CSF, IL-4, IL-3, and/or TNF) or in vivo are considered to be immature dendritic cells. Two different in vitro differentiation pathways have been described in the literature: myeloid and lymphoid (see, for example, Pulendran et al., Proc. Natl. Acad. Sci. USA 96:1036, 1999). These two pathways produce dendritic cells with distinct phenotypic markers. Cells that differentiate along either such pathway are encompassed within the present definition. Immature dendritic cells that are found in tissues are generally characterized by a specialized ability to capture and process antigens, but an inability to act as effective pAPCs for T cell activation. As used herein, the term “immature dendritic cells” cells to which the “immature” designation is typically applied in the art, and also encompasses precursors to such cells. For example, precursor cells that mature in vitro are immature dendritic cells for the purposes of the present invention even though their differentiation into mature dendritic cells may require interaction with microorganisms and/or T cells (e.g., via CD40/CD40 ligand).

“Inducing agents”: are compounds or other agents that induce a pAPC to produce stimulating cytokines. For example, if it is desired that a pAPC secrete Th1 stimulating cytokines, then factors such as LPS, CD40, CD40 ligand, BCGs, oligonucleoties containing CpG motifs, TNFα, and microbial extracts such as preparations of Staphylococcus aureus, heat killed Listeria, modified cholera toxin, etc. can act as inducing agents (“Th1 inducing agents”). If instead it is desired that a pAPC secrete Th2 stimulating cytokines, then other factors (e.g., factors that induce IL-4 expression or inhibit IL-12 expression) can act as inducing agents (“Th2 inducing gents”).

“Isolated”: means separated from at least one of the components with which the isolated entity or compound is associated in nature.

“Mature dendritic cells”: as that term is used herein, are dendritic cells that are capable of acting as effective pAPCs for T cell activation but that are no longer capable of efficiently capturing and processing antigen. In nature, such cells are generated when stimulated immature dendritic cells migrate into the lymphoid organs. For the purposes of the present invention, however, the mechanism by which mature dendritic cells are generated is not relevant. Such cells may be produced in vivo or in vitro, for example through exposure of immature dendritic cells to one or more cytokines such as TNFα, GM-CSF, IL-3, IL-4, followed by exposure to CD40 ligand.

“Native peptide sequence”: as used herein, refers to any peptide sequence that is found in nature. For example, a native peptide sequence has not been modified by recombinant technology.

“Peptide”: refers to a chain of at least four amino acids or amino acid mimics, but can vary in length having an upper limit of about thirty or more amino acids. The term includes peptides in their neutral (uncharged) forms or in forms which are salts, and either free of modifications such as glycosylation, side chain oxidation, phosphorylation or containing these modification, subject to the condition that the modification not destroy the biological activity of the peptides as herein described.

“Primary immune response”: refers to the initial activation of immune system cells when they encounter or recognize a particular antigen for the first time. Immune system cells that have undergone a primary response and have been activated against a particular antigen are said to be “primed” to that antigen.

“Recombinant technology”: as that term is used herein, refers to methods available in the art of manipulating DNA or RNA molecules so that portions of such molecules that are not linked to one another become linked to each other through manipulations selected by a researcher.

“Secondary immune response”: refers to a second or subsequent stimulation of primed cells by re-exposure to antigen.

“Therapeutically effective dose”: means an amount sufficient to elicit the desired response to a particular antigen or composition of antigens, or to at least partially arrest symptoms of the disease and its complications. Amounts effective for this use will depend on, e.g., the nature of the composition (e.g., peptide, cell, etc.), the manner of administration, the stage and severity of the disease being treated, the weight and general state of health of the patient and the judgement of the prescribing physician.

### DESCRIPTION OF CERTAIN PREFERRED EMBODIMENTS OF THE INVENTION

As mentioned above, the present invention encompasses the recognition that immune system responses to particular antigens can be controlled through regulation of the context in which the immune system encounters the antigen. In particular, the invention provides techniques and reagents for biasing a helper T cell response away from either the Th1 or the Th2 pathway.

Those of ordinary skill in the art will appreciate that the direction in which the Th1 vs Th2 response choice is to be influenced will depend upon the particular application in which the inventive techniques and reagents are being employed. Preferably, the inventive techniques and reagents are applied to allergic or autoimmune disorders, as described more fully below.

Those of ordinary skill in the art will further appreciate that the inventive techniques and reagents may be applied to any subject in which it is desirable to avoid or limit an undesirable immune system response. Preferably, the subject is a mammalian individual; more preferably, the subject is human.

Inappropriate Immune Responses

Autoimmune Diseases

According to recent medical literature, autoimmunity (i.e., the production of antibodies against self antigens) is probably a normal event. Autoimmune diseases develop when autoimmune reactions cause tissue damage in an individual. Chronic autoimmune diseases are reported to afflict 50 million Americans.

Damage from autoimmune reactions results when the immune system inappropriately initiates or maintains a response against a self antigen. Certain autoimmune disorders (e.g., autoimmune thyroid disease; multiple sclerosis) have been shown to involve elevated levels of Th1 cytokines in the targeted tissues (see, for example, Romagnani, Clin. Immunol. Immunopathol. 80:225, 1996; Romagnani et al., The Th1/Th2 Paradigm in Disease, R. G. Landes Co., Austin/Springer-Verlag, 1997; Romangnani, Immunol. Today 18:263, June 1997, each of which is incorporated herein by reference). Accordingly, one aspect of the present invention teaches that it is desirable to treat individuals suffering from certain autoimmune diseases by exposing their immune cells to one or more relevant auto-antigens in a context that reduces the Th1 response to the antigen, perhaps by inducing a Th2 response. Another aspect of the invention involves treating individuals who may be susceptible to one or more autoimmune diseases, but have not yet developed clinical symptoms of such disease, in order to “vaccinate” them against the disease by biasing their immune response to disease-triggering antigen toward Th2 rather than Th1. The presents invention, as described herein, provides compositions and methods to achieve these goals.

The teachings and methods of the present invention may be applied to any autoimmune disease including, for example, ankylosing spondylitis, acute anterior uvelitis, Goodpasture's syndrome, multiple sclerosis, Graves' disease, myasthenia gravis, systemic lupus erythematosus, insulin-dependent diabetes mellitus, rheumatoid arthritis, pemphigus vulgaris, Hashimoto's thyroiditis, experimental allergic encephalomyelitis, experimental autoimmune uveorenitis, Crohn's disease, mixed connective tissue disease, scleroderma, Sjorgen's syndrome, autoimmune hemolytic anemia, autoimmune thrombocytopenic purpura, acute rheumatic fever, mixed essential cryoglobulinemia, etc. All that is required is that one or more of the relevant autoantigens be known, so that they may be prepared for presentation to the individual's immune system cells as described herein. Those of ordinary skill in the art will appreciate that relevant autoantigens are already known for a wide range of autoimmune diseases. The following Table presents just a few examples of known autoantigens thought to be involved in the pathology of autoimmune diseases:

TABLE

| DISORDER | AUTOANTIGEN |
|---|---|
| autoimmune hemolytic anemia | Rh blood group antigens |
| | I antigen |
| autoimmune thrombocytopenic | platelet integrin GpIIb:IIIa |
| purpura | |
| Goodpasture‘s syndrome | non-collagenous domain of basement |
| | membrane collagen type V |
| pemphigus vulgaris | epidermal cadherin |
| acute rheumatic fever | cardiac muscle |
| mixed essential cryoglobulinemia | rheumatoid factor IgG complexes |
| systemic lupus erythrmatosus | DNA |
| | histones |
| | snRNP |
| | scRNP |
| insulin-dependent diabetes mellitus | pancreatic β-cell antigen |
| rheumatoid arthritis | synovial joint antigen |
| multiple sclerosis | myelin basic protein |
| | proteolipid protein |
| | myelin oligodendrocyte glycoprotein |
| experimental autoimmune | myelin basic protein |
| encephalomyelitis | proteolipid protein |
| | myelin oligodendrocyte glycoprotein |

Allergy

It is estimated that allergic disorders affect 20-30% of the general population. Current treatments for such disorders attempt to desensitize individuals to specific allergens (e.g., pollen, food allergens, drug allergens, workplace allergens, etc.) against which they have been demonstrated to react. Typically, attempts are made to “vaccinate” a sensitive individual against a particular allergen by periodically injecting or treating the individual with a crude suspension of the raw allergen. The goal is, through controlled administration of known amounts of antigen, to modulate the IgE response mounted in the individual. If the therapy is successful, the individual's IgE response is diminished, or can even disappear. However, the therapy requires several rounds of vaccination, over an extended time period (3-5 years), and very often does not produce the desired results. Moreover, certain individuals suffer anaphylactic reactions to the vaccines, despite their intentional, controlled administration. Clearly, there is a need for an improved system for treating allergic reactions.

The present invention provides such an improved system. In particular, the invention comprises an allergy treatment that involves regulating the context in which an APC presents antigen to one or more immune system cells, in order to limit any Th2/IgE response that could lead to anaphylaxis, for example by promoting a Th1 reaction to the antigen. Furthermore, certain preferred embodiments of the invention, discussed in more detail below, provide methods and reagents that inhibit a Th2 response in an individual without exposing the individual to antigen, and therefore without risking the development of an anaphylactic reaction in the individual.

Compositions and Methods for Regulation of Antigen Presentation

In general, the present invention provides methods and reagents for controlling the context in which an antigen is presented to the immune system. In some embodiments of the invention, this control is effected through modulation of the environment in which an APC, preferably a pAPC, encounters antigen, so that maturation/activation of the APC is influenced in a predetermined manner and the APC, or another cell, is induced to express a desired array of cytokines. The desired cytokine array is selected to bias the response of T cells to which the APC or pAPC presents antigen away from either a Th1 response or a Th2 response. Alternatively or additionally, the environment in which an antigen-loaded APC or pAPC encounters a population of T cells may be modulated (e.g., by altering the array of cytokines produced by the APC or pAPC), so that the Th1/Th2 decision is affected at that point.

In some embodiments of the invention, the environment in which the pAPC encounters antigen, and/or the environment in which the antigen-loaded pAPC encounters the T helper cell, is modulated in vivo; in others, such modulation occurs ex vivo. Each of these different categories of embodiments is discussed individually below.

Modulation of pAPC/antigen and/or pAPC/T Cell Encounter in vivo

In certain embodiments of the invention, control over the immune system response to a particular antigen is exerted through the in vivo administration of one or more cytokines or inducing agents concurrently with exposure of pAPC to antigen and/or to T cell.

It will be appreciated that the present discussion applies to pAPC encounter with or display of any antigen, be it a self-antigen (whether normal or disease-related), an infectious antigen (e.g., a microbial or viral antigen), or some other foreign antigen (e.g., a food component, pollen, etc.). The discussion also applies to collections of more than one antigen, so that immune responses to multiple antigens may be modulated simultaneously. Moreover, the discussion applies to any of a variety of different formulations of antigen, as the antigen is presented to the individual being treated in accordance with the present invention.

For example, the antigen may be in a “natural” form in that no human intervention was involved in preparing the antigen or inducing it to enter the environment in which it encounters the pAPC. Alternatively or additionally, the antigen may comprise a crude preparation, for example of the type that is commonly administered in a conventional allergy shot. The antigen may alternatively be substantially purified, preferably being at least about 90% pure.

Where the antigen is a polypeptide or protein antigen, provision of the antigen may comprise provision of a gene encoding the antigen, so that expression of the gene results in antigen production either in the individual being treated or in another expression system (e.g., and in vitro transcription/translation system or a host cell) from which expressed antigen can be obtained for administration to the individual. Techniques for generating nucleic acids including an expressible gene, and for introducing such nucleic acids into an expression system in which any protein encoded by the expressible gene will be produced, are well established in the art (see, for example, Sambrook et al., Molecular Cloning: A Laboratory Manual, 2nd Ed., Cold Spring Harbor Laboratory Press, Cold Spring Harbor, N.Y., 1989, incorporated herein by reference). These same techniques allow the ready production of fusion proteins, in which portions of sequence from a first polypeptide (e.g., a first antigen) are covalently linked to portions of sequence from a second polypeptide (e.g., a second antigen, a signal sequence, a transmembrane domain, a purification handle, etc.) by means of a peptide bond. Those of ordinary skill in the art will appreciate the diversity of such fusion proteins for use in accordance with the present invention. Recombinant techniques further allow for the ready modification of the amino acid sequence of polypeptide or protein antigens, by substitution, deletion, addition, or inversion of amino acid sequences.

Where the antigen is a peptide, it may be generated, for example, by proteolytic cleavage of isolated proteins. Any of a variety of cleavage agents may be utilized including, but not limited to, pepsin, cyanogen bromide, trypsin, chymotrypsin, etc. Alternatively, peptides may be chemically synthesized, preferably on an automated synthesizer such as is available in the art (see, for example, Stewart et al., Solid Phase Peptide Synthesis, 2d. Ed., Pierce Chemical Co., 1984; see also Example 2). Also, recombinant techniques may be employed to create a nucleic acid encoding the peptide of interest, and to express that peptide under desired conditions (e.g., in a host cell or an in vitro expression system from which it can readily be purified).

The antigen employed in accordance with the present invention may be a naturally-occurring compound or may alternatively have a structure that is distinct from any naturally-occurring compound. In certain embodiments of the invention, the antigen is a “modified antigen” in that the antigen has a structure that is substantially identical to that of a naturally-occurring antigen but that includes one or more deviations from the precise structure of the naturally-occurring compound.

For instance, where the naturally-occurring antigen is a protein or polypeptide antigen, a modified antigen as compared with that protein or polypeptide antigen would have an amino acid sequence that differs from that of the naturally-occurring antigen in the addition, substitution, or deletion of one or more amino acids, and/or would include one or more amino acids that differ from the corresponding amino acid in the naturally-occurring antigen by the addition, substitution, or deletion of one or more chemical moieties covalently linked to the amino acid. Preferably, the naturally-occurring and modified antigens share at least one region of at least 5 amino acids that are at least approximately 75% identical. Those of ordinary skill in the art will appreciate that, in comparing two amino acid sequences to determine the extent of their identity, the spacing between stretches (i.e., regions of at least two) of identical amino acids need not always be precisely preserved. It is generally preferred that naturally-occurring and modified protein or polypeptide antigens show at least approximately 80% identity, more preferably 85%, 90%, 95%, or greater than 99% identity in amino acid sequence for at least one region of at least 5 amino acids. Often, it will be preferable for a much longer region (e.g., 10, 20, 50, or 100 or more amino acids) of amino acid sequence to show the designated degree of identity.

To mention but one preferred embodiment of modified antigens for use in accordance with the present invention, U.S. patent application Ser. No. 09/247,406, entitled “Method for Altering Undesirable Immune Responses to Proteins” and filed on Feb. 10, 1999 (incorporated herein by reference) describes modified antigens, and processes for generating and/or identifying them, against which a less undesirable immune system response is mounted than would be mounted against the naturally-occurring antigen to which they are related. For example, this application describes modified protein antigens in which one or more IgE binding sites have been removed so that the modified antigens elicit less of an undesirable allergic reaction than do their naturally-occurring counterparts.

The amount of antigen to be employed in any particular composition or application will depend on the nature of the particular antigen and of the application for which it is being used, as will readily be appreciated by those of ordinary skill in the art. In general, larger amounts of antigen are useful for inducing Th1 responses, smaller amounts for inducing Th2 responses.

The cytokine(s) or inducing agent(s) to be administered is/are selected, of course, to reduce production of a Th1 or Th2 response, depending on the particular application involved, as discussed above. One preferred method of reducing a Th1 or Th2 response is through induction of the alternative response. Cytokines that, when expressed during antigen presentation to a T cell, induce a Th1 response in T cells (i.e., “Th1 stimulating cytokines”) include IL-12, IL-2, 1-18, IL-1 β or fragments thereof, IFNα, and/or IFNγ, etc.; Th2 stimulating cytokines include IL-4. Inducing agents that prompt the expression of Th1 stimulating cytokines include factors such as LPS, CD40, CD40 ligand, oligonucleoties containing CpG motifs, TNFα, and microbial extracts such as preparations of Staphylococcus aureus, heat killed Listeria, and modified cholera toxin, etc.; inducing agents that prompt the expression of Th2 stimulating cytokines include agents that induce IL-4 expression by T cells or other cells, as well as agents that suppress IL-12 expression by pAPC.

Cytokines or inducing agents may be provided as impure preparations (e.g., isolates of cells expressing a cytokine gene, either endogenous or exogenous to the cell), but are preferably provided in purified form. Purified preparations are preferably at least about 90% pure, more preferably at least about 95% pure, and most preferably at least about 99% pure. Alternatively, genes encoding the cytokines or inducing agents may be provided, so that gene expression results in cytokine or inducing agent production either in the individual being treated or in another expression system (e.g., an in vitro transcription/translation system or a host cell) from which expressed cytokine or inducing agent can be obtained for administration to the individual.

Where both cytokine/inducing agent and antigen are to be delivered to an individual, they may be provided together or separately. For example, both compounds may be associated by means of a common encapsulation device or by means of physical association such as covalent linkage, hydrogen bonding, hydrophobic interaction, van der Waals interaction, etc. In certain preferred embodiments of the invention in which both compounds are provided together, genes encoding both are provided. For example, genes for both may be provided as part of the same nucleic acid molecule. In some embodiments, this nucleic acid molecule may be prepared so that both factors are expressed from a single gene, as a fusion protein in which the cytokine or inducing agent and the antigen are covalently linked to one another via a peptide bond. Alternatively or additionally, the genes may be linked to the same or equivalent control sequences, so that both genes become expressed within the individual in response to the same stimuli. A wide variety of different control sequences, active in different host cells under different conditions is available in the art. Any such control sequences, including constitutive control sequences, inducible control sequences, and repressible control sequences, may be used in accordance with the present invention, though inducible or repressible sequences are particularly preferred for applications in which additional control over the timing of gene expression is desired.

Coordinate control is particularly desirable where one or more of the cytokines, inducing agents, or antigens being employed is a heterodimeric compound (e.g., IL-12). In such cases, it will generally be desirable to express both dimmer components at comparable levels, preferably under control of the same regulatory elements. Also, fusions may be made with one or both dimmer components.

It will be appreciated by those of ordinary skill in the art that the inventive administration of cytokine and/or antigen may optionally be combined with the administration of any other desired immune system modulatory factor such as, for example, an adjuvant or other immunomodulatory compound. Hundreds of different adjuvants are known in the art and could be employed in the practice of the present invention. Particularly preferred are those that induce IL-12 production, including microbial extracts such as fixed Staphylococcus aureus, Streptococcal preparations, Mycobacterium tuberculosis, lipopolysaccharide (LPS), listeria monocytogenes, toxoplasma gondii, leishmania major, etc.

Modulation of pAPC/antigen Encounter ex Vivo

In certain preferred embodiments of the invention, control over immune responses is exerted by modulation of a pAPC/antigen encounter ex vivo (e.g., in vitro). In particular, isolated pAPCs are provided, and are exposed to antigen under conditions that favor antigen uptake and presentation by the pAPC in a manner likely to bias a subsequent response by helper T cells that come into contact with the pAPC away from a Th1 or Th2 response, as desired. It will be appreciated that the strategy described herein would allow the reduction of an undesirable Th1 or Th2 response in an individual without risking exposure of the individual to the relevant antigen, and therefore without the risk that an anaphylactic reaction or other untoward response will be induced in that individual.

Those of ordinary skill in the art will appreciate that the principles of the present invention are applicable to any pAPC or combination of pAPCs. Macrophages readily ingest microorganisms via receptors that recognize microbial components. Ingestion of a microorganism induces expression of MHC class II molecules, and of co-stimulatory factors, by the macrophage, so that effective antigen presentation by macrophages may be functionally limited to infectious rather than innocuous antigens. Macrophages are also potent expressors of IL-12 and therefore are particularly useful for the stimulation of Th1 responses. Like macrophages, B cells are induced to express MHC class II molecules and co-stimulatory factors when they take up and process antigen. B cells, however, are not known to produce IL-12 and therefore may not be able to induce a Th1 response. Such cells are probably useful only in the induction of a Th2 response.

Dendritic cells are particularly preferred for use as pAPC in accordance with the present invention. Dendritic cells are specialized pAPCs found in small numbers in various tissues throughout the body; they can efficiently capture all types of antigens, including soluble proteins (e.g., protein allergens). Dendritic cells express high levels of MHC I and II molecules, as well as co-stimulatory molecules, and are thought to be essential for the initial activation of T cells against a particular antigen. Dendritic cells, like macrophages, and B cells, are also capable of presenting antigen to already-primed T cells, thereby stimulating a secondary immune response.

Dendritic cells originate as precursor cells found in the blood and bone marrow (for review, see Cela et al., Curr. Op. Immunol. 9:10, 1997). When such precursor cells become exposed to antigen, and/or antigen-induced inflammation, they are induced to mature. Maturation involves the expression of high levels of MHC and co-stimulatory molecules. In vivo, this process also involves migration to the vicinity of lymphatic organs (e.g., lymph nodes, spleen, thymus, etc.), where the mature cells present their displayed antigen fragments to the T and B cells that they encounter. At an intermediate stage, immature dendritic cells are observed that are characterized by strong antigen capture and processing capabilities but low ability to activate or stimulate T cells.

Dendritic cells may be matured in vitro by exposing precursor cells to cytokines such as GM-CSF, IL-3, and/or IL-4. Two distinct types of dendritic cells are induced under these conditions, differing in cell surface marker expression, capacity to capture antigen, production of cytokine, and cellular function. Th1 or Th2 responses can be induced by these distinct types of dendritic cells, since one type secretes IL-12 and the other induces IL-4 expression in T cells.

In preferred embodiments of the invention, dendritic cells or dendritic cell precursors are isolated from an individual, preferably but not necessarily the individual to be treated in accordance with the present invention, and are exposed to antigen in vitro. A variety of techniques are available in the art for isolating dendritic cells or dendritic cell precursors from individuals (see, for example, Alijagic et al., Eur. J. Immunol. 25:3100, 1995; Bender et al., J Immunol. Met. 196:121, 1996; Caux et al., Nature 360:258, 1992; DeMatos et al., Cell.

Immunol., 1998; Inaba et al., J. Exp. Med. 180:83, 1994; Sallusto et al., J. Exp. Med. 179:1109, 1994; Szabolcs et al., Blood 87:4520, 1996; Tjandrawan et al., J. Immunother., 1998; Zorina et al., J. Immunother. 16:247, 1994; Engleman et al., WO 95/15540; each of which is incorporated herein by reference). Typically, cells are isolated from bone marrow or peripheral blood samples, and are propagated and/or differentiated in vitro according to established techniques. Different techniques are utilized depending on the type of dendritic cell to be generated during the maturation process. Regardless, all current techniques include a step of treating the maturing cells with CD40 ligand, after which step the cells are competent to induce Th1 or Th2 responses in T cells in vivo.

If desired, the dendritic cell phenotype may be confirmed using standard techniques such as flow cytometry, or other approaches to detecting markers specific to dendritic cells (see, for example, Example 5). Alternatively or additionally, flow cytometry or other techniques may be used to isolate particular subsets of dendritic cells useful in accordance with a relevant application of the present invention.

Techniques are also available for the exposure of isolated dendritic cells to selected antigens in vitro. For example, recent reports have described loading isolated dendritic cells with particular cancer-associated antigens, either by pulsing the cells with antigen in vitro or by transducing them in vitro with nucleic acid encoding the antigen so that they express the antigen themselves (see, for example, Hsu et al., Nature Med. 2:52, 1996; Condon et al., Nature Med. 2:1122, 1996; Okada et al., Int. J. Cancer 78:196, 1998; DeMatos et al., J. Surg. Onc. 68:79, 1998; Nestle et al., “Vaccination of melanoma patients with peptide- or tumor lysate-pulsed dendritic cells” Nature Med., 4:328-32, 1998.; Mayorodomo et al., Nature Med. 1: 1297, 1995; Tüting et al., “Autologous Human Monocute-Derived Dendritic Cells Genetically Modified To Express Melanoma Antigens Elicit Primary Cytotoxic T-Cell Responses In Vitro: Enhancement By Cotransfection of Genes Encoding The Th1-Biasing Cytokines IL-12 and IFN-α¹” J. ImmunoL. 160(3):1139-47, 1998; Kundu et al., Aids Res. Hum. Retrovir. 14:551, 1998; each of which is incorporated herein by reference). The goal of these studies has been to prepare dendritic cells that, when re-introduced into the individual from whom they were isolated, will induce a T-cell response in that individual, thereby effectively immunizing the individual against the relevant cancer-associated antigen or antigens involved. Thus, the idea has been to induce an immune response against the selected antigen (or antigens); prior to the present invention, there was no teaching or suggestion of methods or reagents utilizing isolated dendritic cells that allow for the controlled modulation of a Th1/Thi2 decision in a responding helper T cell population, so as to alter the nature of immune response induced in an individual upon exposure of a particular antigen.

According to the present invention, isolated pAPCs are exposed to antigen in vitro according to any known technique. As above, any formulation of antigen may be employed; alternatively or additionally, the pAPCs may be induced to produce the antigen themselves, for example through transfection with a nucleic acid encoding a protein antigen, or induction of an endogenous gene encoding the antigen or an enzyme responsible for producing the antigen. Also, multiple antigens may be provided, if desired.

The timing of exposure of isolated pAPC to antigen can be varied. In certain embodiments of the invention, for example, pAPCs (or pAPC precursors) are isolated, are expanded and matured in vitro, and are then delivered to an individual in combination with antigen, so that the pAPCs are initially exposed to antigen during the delivery process. In other embodiments, pAPCs are expanded in vitro in the presence of antigen for a period of time, according to known techniques (see, for example, Hsu et al., Nature Med. 2:52, 1996; Condon et al., Nature Med 2:1122, 1996; Okada et al., Int. J. Cancer 78:196, 1998; DeMatos et al., J. Surg. Onc. 68:79, 1998; Nestle et al., Nature Med., 4:328-32, 1998.; Mayorodomo et al., Nature Med. 15 1:1297, 1995; T\ddot{υ}ting et al., J. Immunol. 160(3):1139-47, 1998; Kundu et al., Aids Res. Hum. Retrovir. 14:551, 1998; each of which is incorporated herein by reference).

As above, the amount of antigen will vary, as understood by those of ordinary skill in the art, depending on the nature of the antigen and on the particular application. In general, larger amounts of antigen are employed to induce Th1 responses than Th2 responses.

Concurrently with, exposure to antigen, the isolated pAPCs may optionally be exposed to one or more cytokines or inducing agents in order to induce the pAPCs to present the antigen in a fashion that will inhibit the undesirable response from an encountered T cell or T cell population.

As above, any formulation of cytokine or inducing agent may be employed, including for example, crude preparations, purified preparations, and recombinant preparations. Alternatively or additionally, the pAPCs themselves may be induced to express the relevant cytokines or inducing agents, either by transfection with a nucleic acid encoding a particular cytokine or inducing agent gene, or activation of an endogenous copy of such a gene. Those of ordinary skill in the art will appreciate that the choice of cytokine or inducing agent to be employed will in part be dictated by the details of the exposure. For example, it may be preferable to expose pAPC to inducing agents rather than cytokines if compounds themselves are being administered; if nucleic acids are being administered, it is generally preferred that those nucleic acids encode cytokines rather than inducing agents.

Isolated pAPCs that have been induced to present the desired antigen, and preferably also to express certain selected cytokines, are delivered to the subject. Such cells are preferably delivered in combination with one or more cytokines selected to encourage any helper T cell with which the pAPCs come into contact to adopt the appropriate response. Again, any formulation of cytokine may be employed, and either stimulating or upstream cytokines, or both, may be used.

Delivery of Compositions

As described above, various of the inventive approaches involve delivery of one or more compounds in vivo or in vitro, to a reacting system. In each case, any available delivery means may be employed. Those of ordinary skill in the art will appreciate that the preferred delivery means may vary depending on the form of composition being delivered. For example, compositions including cells (e.g., pAPC), are preferably delivered by injection. Those of ordinary skill in the art will also appreciate that known adjuvants or other immunomodulatory compounds may always be employed in combination with (or as part of) the inventive compositions. Certain particularly preferred means of delivering inventive compositions are discussed in more detail here.

Where compositions are being delivered to a patient, they may be formulated for parenteral, topical, oral, nasal (e.g., by inhalation), or local administration. It is often preferred that such compositions be injected. However, any mode of delivery that accomplishes the exposure of delivered materials to relevant target tissues is acceptable. For example, where isolated pAPCs are being (re-)introduced into an individual, the eventual target tissue for those APCs is lymphatic tissue. Accordingly, they may be injected, for example, into the arm or other general site, or may alternatively be injected directly into lymphatic tissue (e.g., lymph nodes, spleen, etc.), or in close proximity thereto. Alternatively or additionally, they may be applied, for example by spraying, onto mucous membranes or other convenient tissues. Mucosal application is particularly useful for embodiments of the invention relating to inflammatory responses that occur within such tissues (e.g., asthmatic responses and certain allergic reactions, and is preferably utilized for soluble components (e.g., not for cells).

In other preferred embodiments of the invention, compositions are delivered in the context of an encapsulating device. Whether in vivo or in vitro delivery is involved, encapsulation can provide a variety of advantages, including, for example, the ability to maintain various elements of a provided composition in close proximity in order to maximize the probability that the elements will be encountered together by components of the site to which they are delivered. For example, it may be desirable to encapsulate antigen with a stimulating cytokine or an inducing agent prior to delivering the antigen and cytokine or agent to a pAPC in vivo or in vitro, so that the pAPC encounter the antigen and the cytokine together.

In preferred embodiments of the invention, the encapsulated compositions include one or more of antigen(s), cytokine(s), inducing agent(s), and gene(s) encoding antigen and/or cytokine or inducing agent. It may even be possible to include one or more pAPC in the encapsulated composition, if desired.

Encapsulation can also provide the advantage of stabilizing components protected within the encapsulation device. For example, to the extent that one or more elements of the encapsulated composition could otherwise be destroyed during the delivery process, it may be desirable to encapsulate the components in order to help preserve their integrity. Furthermore, particularly where the composition being delivered includes an antigen that is being delivered to a sensitive individual, encapsulation may help minimize the risk that exposure to the antigen will induce an undesirable, e.g., anaphylactic, immune response in that individual prior to (or, worse yet, instead of) the operation of the inventive concepts and techniques. Also, to the extent that encapsulated components are formulated along with additional reagents, such as binders etc., increased flexibility of formulation is provided when those reagents will not be exposed to the immune system.

To the extent that encapsulated materials (e.g., antigens, cytokines or inducing agents) may have undesirable toxicities, at least when distributed systemically, encapsulation may provide the additional advantage of limiting exposure of the individual to which the composition is administered to the toxic materials, at least on a systemic basis.

Where it is desired that the encapsulated materials be taken up by particular cells, it may be desirable to include within the encapsulating device an agent that stimulates such uptake. For example, targeting agents useful to direct the encapsulation device to the cells of interest, as discussed below, may be employed.

Any available encapsulation means may be employed in the practice of the present invention so long as (i) the encapsulated compounds retain a sufficient level of their biological activity to be effective in accordance with the present invention; and (ii) the encapsulation device itself does not elicit a substantial undesirable immune reaction. For example, compositions may be encapsulated within alginate devices, liposomes, chitosan, etc. (see, for example, WO 98/33520, incorporated herein by reference).

Whether or not the inventive compositions are encapsulated when they are administered in vivo or in vitro, it may be desirable to associate them with a targeting agent that will ensure their delivery to a particular desired location. For example, an antigen and cytokine or inducing agent could be encapsulated together in a device that includes a targeting entity that facilitates delivery of the encapsulated components to pAPCs in vivo and/or in vitro. Such an approach might encourage the pAPC to take up the antigen and display it in a desired context, likely to bias responding T cells as discussed herein. In particular, compounds or compositions could be targeted to dendritic cells or macrophages via association with a ligand that interacts with an uptake receptor such as the mannose receptor or an Fc receptor. Compounds or compositions could be targeted to other pAPCs via association with a ligand that interacts with the complement receptor. Compounds or compositions could be specifically directed to dendritic cells by association with DEC205, a mannose-like receptor that is specific for these cells.

Alternatively or additionally, encapsulated compositions could be targeted to particular vesicles within pAPCs. Those of ordinary skill in the art will appreciate that any targeting strategy should allow for proper uptake and processing of antigen by the pAPCs.

An alternative targeting strategy, that could be employed with either an encapsulated or a non-encapsulated composition, is to associate the composition with an Ig molecule, or portion thereof. Ig molecules are comprised of four polypeptide chains, two identical “heavy” chains and two identical “light” chains. Each chain contains an amino-terminal variable region, and a carboxy-terminal constant region. The four variable regions together comprise the “variable domain” of the antibody; the constant regions comprise the “constant domain”. The chains associate with one another in a Y-structure, in which each short Y arm is formed by interaction of an entire light chain with the variable region and part of the constant region of one heavy chain, and the Y stem is formed by interaction of the two heavy chain constant regions with one another. The heavy chain constant regions determine the class of the antibody molecule, and mediate the molecule's interactions with class-specific receptors on certain target cells; the variable regions determine the molecule's specificity and affinity for a particular antigen.

Class-specific antibody receptors, with which the heavy chain constant regions interact, are found on a variety of different cell types and are particularly concentrated on pAPCs, including dendritic cells. According to the present invention, inventive compositions, and particularly antigen-containing compositions, may be targeted for delivery to pAPCs through association with an Ig constant domain. In one embodiment, an Ig molecule is isolated whose variable domain displays specific affinity for the antigen to be delivered, and the antigen is delivered in association with the Ig molecule. The Ig may be of any class for which there is an Ig receptor, but in certain preferred embodiments, is an IgG. Also, it is not required that the entire Ig be utilized; any piece including a sufficient portion of the Ig heavy chain constant domain i sufficient. Thus, Fc fragments and single-chain antibodies may be employed in the practice of the present invention.

In one embodiment of the invention, a polypeptide or protein antigen is prepared as a fusion molecule with at least an Ig heavy chain constant region (e.g., with an Fc fragment), so that a single polypeptide chain, containing both antigen and Ig heavy chain constant region components, is delivered to the individual (or system). This embodiment allows increased flexibility of antigen selection because the length and character of the antigen is not constrained by the binding requirements of the Ig variable domain cleft. In particularly preferred versions of this embodiment, the antigen portion and the Fc portion of the fusion molecule are separated from one another by a severable linker that becomes cleaved when the fusion molecule is taken up into the pAPC. A wide variety of such linkers is known in the. Fc fragments may be prepared by any available technique including, for example, recombinant expression (which may include expression of a fusion protein) proteolytic or chemical cleavage of Ig molecules (e.g., with papain), chemical synthesis, etc.

It will be appreciated that, in certain cases, compositions are to be delivered in accordance with the present invention for therapeutic purposes. In therapeutic applications, compositions of the present invention are administered to a patient in a therapeutically effective dose. Therapeutically effective amounts of the compositions of the present invention generally will fall within a broad range for the initial administration (that is for therapeutic or prophylactic initial immunization).

For example, inventive compositions may be utilized alone, or in combination with other available treatments, to desensitize individuals to antigens to which they are allergic. In such applications, inventive compositions may, for example, be injected as described in Nestle et al., (supra) who administer weekly injections of antigen-loaded dendritic cells over a period of one month. A dose of 1×10⁶ cells was administered into a lymphoid organ per injection. In some instances, it may be desirable to administer booster immunizations to encourage a sustained response to the antigen. For example, booster immunizations may be delivered after 2 weeks and thereafter at monthly intervals by the same methods as described above. Administration may vary over weeks, months or years depending upon the patient's response and condition. For instance after successful elimination of allergic reactions, further treatment might be required to maintain desensitization to an allergen or improve other allergic symptoms.

One of ordinary skill in the art will recognize that a wide range of variation in the regimen of administration that delivers inventive compositions to living individuals can be used according to the present invention. Any variation in dose range or administration, (whether preliminary administration or booster), can be determined according to standard techniques by one of ordinary skill in the art and response can be monitored to ascertain the optimal therapeutic dose under particular circumstances. Of course, any therapeutically effective dose of the inventive composition is acceptable according to the present invention.

Assaying Effect of Inventive Treatments

Those of ordinary skill in the art will appreciate that any of a wide variety of assays may be employed to monitor the effects of inventive treatments described herein. For example, it may be desirable to assay the ability of in vitro-promulgated pAPCs to present antigen as desired, and/or to induce the desired response in helper T cells, prior to (or, in certain cases, instead of) introduction of the pAPCs into an individual or other system containing T cells. Any test may be used to accomplish such an assay; Examples 5-9 present certain preferred assays that could be used as tests to analyze the abilities and characteristics of in vitro-promulgated dendritic cells, and/or their appropriateness for administration to selected individuals.

### EXAMPLES

### Example 1

### Isolation of Dendritic Cells from Peripheral Blood

The present Example describes one procedure that can be employed to isolate dendritic cells for use in accordance with the present invention from peripheral blood.

Dendritic cells are prepared form PBMC as described in T\ddot{υ}ting et al., “Autologous Human Monocute-Derived Dendritic Cells Genetically Modified To Express Melanoma Antigens Elicit Primary Cytotoxic T-Cell Responses In Vitro: Enhancement By Cotransfection of Genes Encoding The Th1-Biasing Cytokines IL-12 and IFN-α¹” (J. Immunol., 160(3):1139, 1998) or Nestle et al., “Vaccination of melanoma patients with peptide- or tumor lysate-pulsed dendritic cells” (Nature Med., 4:328-32, 1998). PBMC are isolated from electrophoresed blood of healthy donors by density centrifugation on Ficoll-hypaque gradients (1.077 g/ml; LSM, Organon-Teknika, Durham, N.C.) for 20 min at 2000 rpm at room temperature. After four or five washes in HBSS (Life Technologies, Gaithersburg, Md.) to remove platelets, cells are resuspended at 10⁷/ml in AIM-V medium (Life Technologies) and incubated for I h in 75-cm² tissue culture flasks (37° C., 5% CO₂). Nonadherent cells are gently washed out with HBSS and cryopreserved for use as T cell responders. The remaining plastic-adherent cells are further cultured (37° C., 5% CO₂) in AIM-v medium supplemented with 1000 U/ml rGM-CSF and 1000 U/rIL4 (Schering-Plough). After 7 to10 days, nonadherent cells are harvested. DC generated in this way are 50 to 80% pure based on morphology and the expression of a CD3⁻, CD14⁻, CD16⁻, CD20⁻, CD40⁺, CD80⁺, CD86⁺, MHC class II⁺ immunophenotype as assessed by flow cytometry. Dendritic cells may also be purified further (>95%) by density gradient centrifugation.

Example 2

### Pulsing Dendritic Cells with Antigen

This Example describes one procedure that may be used to load isolated dendritic cells (e.g., cells prepared as described in Example 1) with an antigen.

Dendritic cells can be pulsed with antigen by any of a variety of methods. For example, dendritic cells are exposed to, for example, 50-100 mg/ml of peptide, protein, or lysate, or digests and incubated at room temperature for 2 to 4 hours. Alternatively, incubation can take place at 37° C. for a few hours or overnight (see, for example, Mayordomo et al., Nature Med., 1:1297, 1995); Okada et al., supra; DeMatos et al., supra; T\ddot{υ}ting et al. supra; each of which is incorporated herein by reference.

### Example 3

### Isolating Dendritic Cell Subpopulations Specific for Th1 or Th2 Activation

The present Example describes the isolation of CD8α⁺ and CD8α⁻ dendritic cells subsets, that induce Th1 or Th2 responses, respectively, in responding T cells (see Maldonado-Lopez, et al., J. Exptl. Med. 189:587, Feb. 1, 1999, incorporated herein by reference). Dendritic cells are isolated and pulsed with antigen according to known techniques. Pulsed cells are then incubated with microbeads coupled to anti-CD8α antibody, and are separated according to CD8α expression by two passages over a MACS® column (Miltenyi Biotec). CD8α⁻ dendritic cells are further enriched by incubation with microbeads coupled to anti-CD1c, followed by positive selection over a MACS® column. Alternatively, antigen-pulsed dendritic cells are separated into CD8α⁺ and CD8α⁻ sub-populations by FACS® sorting. Briefly, cells are double-stained for CD11c and CD8α expression using FITC-conjugated N418 and biotin-conjugated anti-CD8α mAb (PharMingen) followed by PE-streptavidin. The cells are gated based on characteristic forward and side light scatter, and two populations (CD11c⁺ CD8α⁻ and CD11c⁺ CD8α⁻ ) are sorted on a FACSVantage® machine (Becton Dickinson).

These sorted, antigen-pulsed cells may be delivered to individuals (e.g., by injection) to induce either a Th1 or a Th2 response, as desired.

### Example 4

### Particle-Mediated Gene Transfer to DC

This Example describes a procedure by which exogenous nucleic acids may be introduced into isolated dendritic cells (e.g., into cells prepared as described in Example 1).

The following protocol is adopted from Tibting et al., supra, incorporated herein by reference. Plasmid DNA encoding peptide, protein antigenic fragment, or cytokine of interest is precipitated onto 2.6 mm gold particles at a density of 2 mg of DNA/mg of particles as previously described (Irvine et al., J. Immunol., 156:23, 1996; Condon et al., Nature Med. 2:1122, 1996). Briefly, gold particles and DNA are resuspended in 100 μl of 0.05 M spermidine (Sigma Chemical Co., St. Louis, Mo.), and DNA is precipitated by the addition of 100 μl of 1 M CaCl₂. Particles are washed in dry ethanol to remove H₂O, resuspended in dry ethanol containing 0.075 mg/ml of polyvinylpyrrolidone (Sigma Chemical Co.), and coated onto the inner surface of Tefzel tubing using a tube loader. The tubing is cut into 0.5-in. segments, resulting in the delivery of 0.5 mg of gold coated with 1 μg of plasmid DNA/transfection with the Accell helium pulse gun. Gold particles, tubing, tube loader, and the Accell helium pulse gun may be provided by Auragen/Geniva (Middletown, Wis.).

Monocyte-derived dendritic cells are transfected in suspension in six-well plates. Dendritic cells are harvested and pelleted by centrifugation; 2×10⁶ cells are resuspended in 20 μl of fresh medium and spread evenly in the center of a well. Cells are bombarded at a pressure of 300 psi of helium, and fresh culture medium was added immediately. Five to ten percent of dendritic cells can be transfected, as assessed using enhanced green fluorescent protein (PEGFP, Clontech, Palo Alto, Calif.) as a reporter gene.

### Example 5

### Injection of Primed Dendritic Cells

The present example describes a procedure by which dendritic cells, which have been isolated and loaded with antigen (i.e., have been primed), for example as described in the prior Examples, may be administered to individuals.

Primed dendritic cells are harvested from in vitro and injected into a noninvolved inguinal lymph node of an allergic individual (i.e., 1×10⁶ cells per injection). Injections are administered on a weekly basis for a month and optionally as a booster thereafter. Booster immunizations may be delivered 2 weeks after the initial desensitization and thereafter in monthly intervals by the same methods as described above.

### Example 6

### Assays for Cell Response

The present Example describes a collection of different techniques that may be employed to determine whether APC or pAPC have responded as expected after exposure to antigen, or whether T cells have responded as expected after exposure to APC-presented (including pAPC-presented) antigen.

ELISA for Expression of Antigen and Cytokines

This assay measures cytokine secretion by primed cells. One million isolated PBMCs are stimulated with a specific antigen(s) (e.g. a peptide, protein or cellular lysate), and control protein for 48-72 hr. Supernatant is collected and analyzed for IFN-g,IL-12, IL4, IL-5 by enzyme-lined immunosorbent assay (ELISA; Endogen, Woburn, Mass.).

Procedures for performing the ELISA can be found in Sambrook et al., supra or Ausubel et al., supra, incorporated herein by reference (see also T\ddot{υ}ting et al., supra).

Flow Cytometry

For immunophenotying, DC or T cell responders are washed in HBSS supplemented with 1% BSA and 0.1% NaN₃ and incubated (30 min at 4° C.) with one of the following monoclonal antibodies under conditions that allow the antibodies to interact with their targets (some of which are cytoplasmic): anti-IL-12 Dr. M. Gately (Hoffman LaRoche, Nutley, N.J.), anti-IFN-g, anti IL-10, PE-conjugated anti-HLA-DR (Becton Dickinson, Mountain View, Calif.) FITC-conjugated anti-CD80 (Ancell, Bayport, Minn.), FITC-conjugated anti-CD86 (PharMingen, San Diego, Calif.), FITC-conjugated anti -CD40 (PharMingen), PE-conjugated anti-CD3 (Becton Dickinson), FITC-conjugated anti-CD4 (Becton Dickinson), PE-conjugated anti-CD8 (Becton Dickinson), FITC-conjugated anti-CD14 (Becton Dickinson), PE-conjugated anti-CD16 (Becton Dickinson), and FITC-conjugated anti-CD20 (Becton Dickinson). DC are also stained with corresponding isotype-matched control monoclonal antibody (PharMingen). Surface expression is analyzed using a FACScan flow cytometer (Becton Dickinson) and LYSIS II software; data are collected on 5,000 to 10,000 viable cells (Titing et al., supra).

Lymphocyte Proliferative Assay

Lymphocyte proliferation assays are performed as described by Valentine et al. (J. Infect. Dis., 173:1336, 1996). Peripheral blood mononuclear cells are stimulated with antigen. Mean [³H] thymidine incorporation is determined and the results are expressed as a stimulation index (SI) over a no antigen or irrelevant antigen control (see also, Kundu et al., supra).

Delayed Hypersensitivity Tests

Delayed hypersensitivity tests (DHT) are performed to assess responses to allergenic fragments. Typically, selected antigens are injected intradermally; injection sites are checked for induration after 48 hr. An induration 5 mm or more in diameter is interpreted as a positive response (see Kundu et al., supra) (see Nestle et al., supra, incorporated herein by reference).

### Example 7

### Detection of in vivo T Cell Response Induced by Administration of Inventive Dendritic Cell Composition

The present Example describes the administration of an inventive dendritic cell vaccine composition to an individual, in this case a mouse, and the subsequent detection of an appropriate T cell response in that individual.

Dendritic cells are isolated and exposed to antigen in vitro as described in Examples 1 and 2. These cells are then injected into a mouse as described in Example 4. T cells are then isolated from the mouse and assayed in vitro for their ability to respond to antigen presented by an APC. Such an ability to respond indicates that the T cells were primed in vivo by exposure to the inventive dendritic cell composition.

Furthermore, the nature of the T cell response is determined by assaying for Th1 or Th2 cytokines as described above in Example 5. The nature of the dendritic cell composition can then optionally be adjusted to achieve optimal T cell activation and desired cytokine secretion.

### Example 8

### Suppression of Existing Th2 Response by Administration of Inventive Compositions

The present Example describes administration of an inventive dendritic cell composition to an individual who has previously mounted a Th2 response to a particular antigen, in order to shift that individual's response to that antigen toward a Th1 response.

Dendritic cells are isolated and exposed to antigen in vitro as described in Examples 1 and 2. These cells are then injected into a subject as described in Example 4, the subject having been selected on the basis of having previously mounted a Th2 response to the antigen (e.g., on the basis of being allergic to the antigen). T cells are then isolated from the subject, and are assayed to detect Th1 cells primed against the antigen.

If the subject to whom the dendritic cell composition is to be administered is a non-human animal and is not known to have mounted a prior Th2 response against the antigen, any one of a variety of known protocols may be employed to generate a Th2 response in that animal. For example, in a murine asthma model, inhaled antigen can generate an ongoing Th2 response. The animal can then be injected with dendritic cells primed with antigen used to induce the asthma. The animal can be monitored for decreases in lung inflammatory responses associated with a Th2 response and decreases in secreted IL-4 and IL-5 from CD4⁺ T cells in the lung, brochoalveolar lavage fluid or draining lymph node. T lymphocytes are subsequently removed from the animal and cultured with antigen presenting cells and a dose range of antigen. Changes in cytokine production by T cells after dendritic cell injections compared to controls are determined. Certain experimental details will of course be apparent to the skilled artisan including the realization that these methods may be modified to test the antigenicity of any other antigen or allergen (see also Adorini et al., Nature 334:623, 1988).

### Example 9

### In vitro Assays for Determining the Nature of a T Cell Response

The present Example describes a collection of assays that could be used to determine the nature of a T cell response mounted after administration of an inventive composition. As mentioned above, expression of cytokines representing a Th1 immune response, for example IFN-g and cytokines representing a Th2 response, for example, IL-4 and IL-5, can be assessed in cell culture supernatant by ELISA using antibodies specific to the particular cytokines of interest (see Example 5). Dendritic cells transfected with expression vectors encoding any combination of IL-12, IFN-a, or IFN-g typically produce 40 to 200 pg of IL-12 (see Example 5).

### Example 10

### Use of Control Allergen to Monitor Induction of Appropriate T Cell Response

Allergic reactions to particular allergens cannot always be detected in humans. Accordingly, the present invention provides a test that utilizes a control allergen, known to evoke a detectable response, as a proxy for the antigen of interest.

In general, the known antigen keyhole limpet hemocyanin (KLH), which is often included in conventional allergy vaccinations as a helper immunogen to recruit CD4⁺ helper T cells, is exposed to pAPCs in vitro, in parallel with a test antigen. Exposed pAPCs are then administered to an individual, and a T cell proliferation assay, as described above, is utilized to assess the ability of the pAPCs to stimulate T cell proliferation in vivo (see Example 5). A positive response to KLH and resulting T cell proliferation indicates that the pAPCs are active and are appropriately presenting antigen in vivo to T cells. Of course one of ordinary skill in the art will recognize that any antigen other than KLH may be used in this assay as long as there is a record of safety. It will be obvious to one skilled in the art that these tests may be carried out in an animal model as well.

Similarly, the success of the inventive protocols can be assessed in vitro by isolating PMBC from individuals who have received inventive pAPCs and control individuals who have not, and assaying for the production of allergen or KLH induced cytokines by ELISA or flow cytometry (see Example 5). A Th1 response can be identified by production of high levels of IFN-g but little to no IL-4/IL-5. A Th2 response can be identified by production of high levels of IL-4 and IL-5 and little IFN-g.

### Example 11

### Assaying Response to Antigen in Vivo

In vivo response to antigen may be assessed by the either the skin prick test or the delayed hypersensitivity test (DHT) (see Example 5). Both the skin prick test and the DHT are measures of in vivo response to antigen which target the skin. The skin prick test measures the release of histamine and other mediators that rapidly produce a wheel and erythema upon intradermal challenge. An allergic reaction to a skin prick test is immediate (approximated 20 minutes). IgE antibodies, (specific to the particular antigen administered by the skin prick), are responsible for this reaction. A decrease in the resulting immune reaction to a skin prick test would indicate a desensitization to the alleged antigen.

The DHT response is an indication of the in vivo Th1 type response and thus takes days to produce a visible immune reaction. A positive response to a DHT would be accompanied by release of IFN-g and other Th1-related cytokines, and would indicate that a Th1 type immune system has been elicited. In allergic individuals it is proposed that the introduction of a strong Th1 response (as indicated by positive DHT test) will inhibit Th2 induced IgE (as indicated in skin prick test). Alternatively, a reduction in the allergic response would indicate successful desensitization of the individual to the specific allergen.

As discussed above, various embodiments of the present invention are particularly useful for their ability to “vaccinate” an individual against an antigen to which that person otherwise is or might be allergic. Successful vaccination can be determined by the presence of an allergen-specific DHT test or skin prick test (if it is deemed safe to administer such a test to an allergic individual).

For example, if, as discussed above in Example 9, KLH is administered simultaneously with the allergen to pAPCs in vitro, a KLH-specific DHT would indicate successful vaccination. If KLH was not coadministered with the test antigen an antigen-specific DHT can be administered if safe.

A reduction in allergen positive skin prick test reaction indicates a modulation of the Th2 induced IgE response and reduced sensitization of the individual to the allergen. The absence of a prick test positive immune response would indicate successful desensitization to the specific allergen. Alternatively, a reduction in levels of allergen specific IgE would indicate a successful desensitization to the specific allergen in the allergic individual. Successful vaccination can be determined by measuring serum total or allergen-specific IgE over time.

Patients may also be monitored for successful desensitization to antigen by comparing data between control and test subjects regarding recurrence of severe allergic symptoms over time or exposure the allergenic substance. Standard statistical analysis are well known in the art and can be found in any college statistical textbook. For example, Student's t-test may be used to evaluate the significance of differences between experimental groups. A P-value of <0.05 is considered significant. A significant decrease in recurrence of severe allergic reactions in individuals administered dendritic cell vaccinations would indicate that the vaccinations are successful in desensitizing allergic individuals to a particular allergen or group of allergens.

### OTHER EMBODIMENTS

Those of ordinary skill in the art will readily appreciate that the foregoing represents merely certain preferred embodiments of the present invention. A wide variety of modifications and alterations may be made without departing from the spirit or scope of the present invention, as set forth in the following claims.

We claim:

1. A method of modulating an immune system response to an antigen, the method comprising steps of:
identifying an individual who has been or will be exposed to an antigen; and
administering to the individual, concurrently with exposure to the antigen, a composition comprising at least one factor selected from the group consisting of cytokines and inducing agents, which factor is selected to bias the individual's immune response to the antigen away from a Th1 or Th2 response in a predetermined manner.

2. The method of claim 1, wherein:
the step of identifying comprises identifying an individual who is allergic to the antigen; and
the step of administering comprises administering a composition comprising at least one factor selected to bias the individual's immune response to the antigen away from a Th2 response.

3. The method of claim 2, wherein:
the step of identifying comprises identifying an individual who has previously mounted a Th2 response to the antigen.

4. The method of claim 2, wherein:
the factor comprises a Th1 stimulating cytokine.

5. The method of claim 2, wherein:
the factor is selected from the group consisting of IL-12, IL-2, IL-18, IL-1β, fragments of IL-1β, IFNα, and IFNγ.

6. The method of claim 2, wherein:
the factor comprises a Th2 stimulating cytokine.

7. The method of claim 2, wherein:
the factor is selected from the group consisting of LPS, CD40, CD40 ligand, BCGs, oligonucleotides containing CpG motifs, TNFα, and microbial extracts.

8. The method of claim 7, wherein:
the microbial extracts are selected from the group consisting of any Staphylococcus aureus preparation, heat killed Listeria, and modified cholera toxin.

9. The method of claim 4, wherein:
the step of administering comprises delivering the factor to the vicinity of T cells.

10. The method of claim 7, wherein:
the step of administering comprises delivering the factor to the vicinity of a pAPC that will internalize and display antigen to T cells.

11. The method of claim 1, further comprising a step of:
administering the antigen to the individual.

12. The method of claim 1, wherein:
the step of administering the antigen comprises administering a crude antigen preparation.

13. The method of claim 11, wherein:
the step of administering the antigen comprises administering a substantially pure antigen.

14. The method of claim 11, wherein:
the antigen is a polypeptide antigen; and
the step of administering the antigen comprises administering a gene encoding the antigen, so that the gene becomes expressed within the individual.

15. The method of claim 14, wherein:
the step of administering comprises administering at least one factor that is a protein, and further comprises delivering the protein factor by administering to the individual a gene encoding that factor.

16. The method of claim 2, wherein:
the steps of administering the antigen and administering the composition are performed together and comprise administering a single nucleic acid construct including genes for antigen and protein factor.

17. The method of claim 4, wherein:
the step of administering the single nucleic acid construct comprises administering a construct in which the antigen gene and protein factor gene are linked to one another so that a single fusion protein, containing both antigen and protein factor, is encoded.

18. The method of claim 2, wherein:
the antigen gene and the factor gene are provided on separate nucleic acid molecules.

19. The method of claim 2 or claim 18, wherein:
the antigen gene and the factor gene are coordinately regulated.

20. The method of claim 1 wherein the factor is administered in association with a targeting agent.

21. The method of claim 11 wherein one or both of the antigen and the factor is encapsulated.

22. The method of claim 11, wherein:
the steps of administering the antigen and administering the composition are performed together and comprise administering the antigen and composition in association with one another.

23. The method of claim 22, wherein:
the antigen and factor are administered in association with a targeting agent.

24. The method of claim 20 or claim 23, wherein:
the targeting agent association occurs by means of an interaction selected from the group consisting of covalent bonds, hydrophobic interactions, van der Waals interactions, and combinations thereof.

25. The method of claim 23, wherein:
the targeting agent is selected from the group consisting of mannose receptor ligand and the Fc receptor ligand.

26. The method of claim 29 wherein:
the targeting agent comprises complement receptor ligand.

27. The method of claim 23, wherein:
the targeting agent comprises DEC205.

28. The method of claim 23, wherein:
the targeting agent comprises a ligand that interacts with a receptor on an intracellular vesicle within a pAPC.

29. The method of claim 23, wherein:
the targeting agent comprises at least the Fc portion of an Ig molecule.

30. The method of claim 23, wherein:
the targeting agent comprises at least the Fc portion of an IgG molecule.

31. The method of claim 22, wherein:
the step of administering comprises encapsulating the antigen and the factor together in a single encapsulation device.

32. The method of claim 22, wherein:
the step of administering comprises encapsulating the antigen and the factor in separate encapsulation devices.

33. The method of claim 31 or 32, wherein:
the step of administering the encapsulation device comprises associating the encapsulation device with a targeting agent.

34. The method of claim 33, wherein:
the targeting agent is selected from the group consisting of mannose receptor ligand and the Fc receptor ligand.

35. The method of claim 33, wherein:
the targeting agent comprises complement receptor ligand.

36. The method of claim 33, wherein:
the targeting agent comprises DEC205.

37. The method of claim 33, wherein:
the targeting agent directs the composition to particular vesicles within pAPCs.

38. The method of claim 33, wherein:
the targeting agent comprises at least the Fc portion of an Ig molecule.

39. The method of claim 33, wherein:
the targeting agent comprises at least the Fc portion of an IgG molecule.

40. The method of claim 22, wherein:
the step of administering comprises providing antigen and factor that are covalently linked to one another.

41. The method of claim 22, wherein:
the step of administering comprises providing antigen and factor that are associated with one another by means of an interaction selected from the group consisting of hydrogen bonds: van der Waals interactions, hydrophobic interactions, and combinations thereof.

42. The method of claim 11, wherein:
the step of administering the antigen comprises administering a modified antigen.

43. The method of claim 42, wherein:
the modified antigen is substantially identical to a naturally-occurring antigen that contains at least one IgE binding site, but differs from that naturally-occurring antigen in that the modified antigen is missing at least one of the IgE binding sites.

44. The method of claim 1, wherein:
the antigen comprises an autoantigen;
the step of identifying an individual comprises identifying an individual who has mounted an undesirable auto-immune response against the antigen; and
the factor is selected to bias the individual's immune response to the antigen away from a Th1 response.

45. The method of claim 44, wherein:
the step of administering comprises administering a Th2 stimulating cytokine

46. The method of claim 44, wherein:
the step of administering comprises administering IL-4.

47. The method of claim 45, wherein:
the step of administering comprises delivering the IL-4 to the vicinity of responding T cells.

48. The method of claim 44, wherein:
the step of administering comprises administering one or more Th2 inducing agents

49. The method of claim 44, wherein:
the step of administering comprises administering an agent that induces IL-4 expression.

50. A method of modulating an immune system response to an antigen, the method comprising steps of:
isolating from an individual one or more pAPC selected from the group consisting of: mature pAPC, immature pAPC, and precursors to pAPC;
exposing the isolated cells to an antigen so that pAPC displaying the antigen are generated, and a pre-determined set of cytokines is expressed.

51. The method of claim 50, further comprising:
administering the antigen-exposed pAPC to a subject whose immune response to the antigen is to be modulated.

52. The method of claim 51, wherein:
the antigen-exposed pAPC are mature pAPC.

53. The method of claim 51, wherein:
the antigen-exposed pAPC are immature pAPC

54. The method of claim 51, wherein:
the pAPC are selected from the group consisting of dendritic cells, B cells, and macrophages.

55. The method of claim 51, wherein:
the pAPC are dendritic cells.

56. The method of claim 51, wherein:
the step of isolating comprises isolating immature dendritic cells from an individual; and
maturing the immature cells in vitro by exposure to one or more compounds selected from the group consisting of: GM-CSF, IL-3, and IL-4.

57. The method of claim 53, wherein:
the step of maturing is performed concurrently with the step of exposing to antigen.

58. The method of claim 50, wherein:
the pre-determined set of cytokines is selected from the group consisting of Th1 cytokines and Th2 cytokines.

59. The method of claim 57, wherein:
the Th1 cytokines are selected from the group consisting of IFNα, and/or IFNγ and the Th2 cytokines are selected from the group consisting of IL-4 and IL-5, etc.

60. The method of claim 50, wherein:
the step of exposing the isolated cells to an antigen comprises exposing the cells to a crude antigen preparation.

61. The method of claim 50, wherein:
the step of exposing the isolated cells to an antigen comprises exposing the cells substantially pure antigen.

62. The method of claim 50, wherein:
the antigen is a polypeptide antigen; and
the step of exposing the isolated cells to antigen comprises exposing the cells to a gene encoding the antigen, so that the gene becomes expressed within the cells.

63. The method of claim 50, wherein:
the step of exposing the cells to antigen comprises contacting the cells with an antigen that is associated with a targeting agent.

64. The method of claim 50, wherein:
the step of exposing the isolated cells to an antigen further comprises exposing the cells to a composition comprising a factor selected from the group consisting of cytokines and inducing agents, which factor is selected to bias an immune response in a subject away from a Th1 or a Th2 response in a pre-determined manner.

65. The method of claim 64, wherein:
the step of exposing comprises exposing the cells to one or more Th1 inducing agents.

66. The method of claim 65, wherein:
the Th1 inducing agents are selected from the group consisting of LPS, CD40, CD40 ligand, BCGs, oligonucleotides containing CpG motifs, TNFα, and microbial extracts.

67. The method of claim 66, wherein:
the microbial extracts are selected from the group consisting of any Staphylococcus aureus preparation, heat killed Listeria, and modified cholera toxin.

68. The method of claim 64, wherein:
the cytokines comprise Th1 stimulatory cytokines.

69. The method of claim 68, wherein:
the cytokines are selected from the group consisting of IL-12, IL-2, IL-18, IL-1β, fragments of IL-1β, IFNα, and IFNγ.

70. The method of claim 64, wherein:
the step of exposing comprises exposing the cells to one or ore Th2 inducing agents.

71. The method of claim 70, wherein:
the Th2 inducing agents are characterized by an ability to induce IL-4 expression in the pAPC.

72. The method of claim 64, wherein:
the cytokines comprise Th2 stimulatory cytokines.

73. The method of claim 64, wherein:
the cytokines comprise IL-4.

74. The method of claim 64, wherein:
the factor is a polypeptide; and
the step of exposing the cells to a composition comprising the factor comprises contacting the cells with a gene encoding the factor.

75. The method of claim 74, wherein: the gene encoding the antigen and the gene encoding the factor are coordinately regulated.

76. The method of claim 74, wherein:
the gene encoding the antigen and the gene encoding the factor are provided on the same nucleic acid molecule.

77. The method of claim 76, wherein:
the gene encoding the antigen and the gene encoding the factor are linked together so that a fusion protein is encoded.

78. The method of claim 74, wherein:
the gene encoding the antigen and the gene encoding the factor are provided on separate nucleic acid molecules.

79. The method of claim 64, wherein:
the one or both of the antigen and factor are associated with a targeting agent.

80. The method of claim 79, wherein:
the association with the targeting agent occurs by means of an interaction selected from the group consisting of covalent bonds, hydrogen bonds, van der Waals interactions, hydrophobic interactions, and combinations thereof.

81. The method of claim 79, wherein:
the targeting agent is selected from the group consisting of mannose receptor ligand and the Fc receptor ligand.

82. The method of claim 79, wherein:
the targeting agent comprises complement receptor ligand.

83. The method of claim 79, wherein:
the targeting agent comprises DEC205.

84. The method of claim 79, wherein:
the targeting agent is capable of targeting to intracellular vesicles within pAPCs.

85. The method of claim 79, wherein:
the targeting agent comprises at least the Fc portion of an Ig molecule.

86. The method of claim 79, wherein:
the targeting agent comprises at least the Fc portion of an IgG molecule.

87. The method of claim 50, wherein:
the antigen is encapsulated.

88. The method of claim 64, wherein:
the step of exposing comprises providing the antigen and factor together in an encapsulation device.

89. The method of claim 64, wherein:
the step of administering comprises providing the antigen and the factor in separate encapsulation devices.

90. The method of claim 87, 88, or 89, wherein:
the step of exposing comprises exposing the cells to the encapsulation device in association with a targeting agent.

91. The method of claim 90, wherein:
the targeting agent is selected from the group consisting of mannose receptor ligand and the Fc receptor ligand.

92. The method of claim 90, wherein:
the targeting agent comprises complement receptor ligand.

93. The method of claim 90, wherein:
the targeting agent comprises DEC205.

94. The method of claim 90, wherein:
the targeting agent is capable of targeting to particular vesicles within pAPCs.

95. The method of claim 90, wherein:
the targeting agent comprises at least the Fc portion of an Ig molecule.

96. The method of claim 90, wherein:
the targeting agent comprises at least the Fc portion of an IgG molecule.

97. The method of claim 64, wherein:
the step of exposing comprises providing antigen and factor that are associated with one another by means of an interaction selected from the group consisting of: covalent bonds, hydrogen bonds, van der Waals interactions, hydrophobic interactions, and combinations thereof.

98. The method of claim 50, wherein:
the step of exposing the antigen comprises exposing the cells to a modified antigen.

99. The method of claim 64, wherein:
the antigen comprises an autoantigen;
the factor is selected to bias the immune response to the antigen away from a Th1 response.

100. The method of claim 99, wherein:
the factor comprises a Th2 inducing agent.

101. The method of claim 99, wherein
the factor comprises an agent that induces IL-4 expression in the pAPC.

102. The method of claim 64, wherein:
the antigen comprises an allergen; and
the factor is selected to bias the immune response to the antigen away from a Th2 response.

103. The method of claim 102, wherein:
the factor comprises a Th1 inducing agent.

104. The method of claim 102, wherein:
the factor is selected from the group consisting of LPS, CD40, CD40 ligand, BCGs, oligonucleotides containing CpG motifs, TNFα, and microbial extracts.

105. The method of claim 104, wherein:
the microbial extracts are selected from the group consisting of any Staphylococcus aureus preparation, heat killed Listeria, and modified cholera toxin.

106. The method of claim 51, wherein:
the step of administering further comprises administering a cytokine selected from the group consisting of Th1 stimulatory cytokines and Th2 stimulatory cytokines to the subject.

107. The method of claim 106, wherein:
the Th1 stimulatory cytokines are selected from the group consisting of IL-12, IL-2, IL-18, IL-1β, fragments of IL-1β, IFNα, and IFNγ and the Th2 stimulatory cytokines are selected from the group consisting of IL-4.

108. The method of claim 51 or claim 101, further comprising:
administering antigen to the subject.

109. A method of modulating an immune system response to an antigen, the method comprising steps of:
isolating from an individual one or more APC selected from the group consisting of: mature pAPC, immature pAPC, and precursors to pAPC;
exposing the isolated cells to an antigen so that mature pAPC displaying the antigen are generated; and
contacting the antigen-exposed pAPC with T cells so that a pre-determined T-cell response is inhibited.

110. The method of claim 109, wherein:
the step of exposing is performed under conditions selected so that mature pAPC displaying antigen is a produced and a pre-determined set of cytokines, selected from the group consisting of Th1 cytokines and Th2 cytokines, is expressed.

111. The method of claim 109 wherein:
the pre-determined T cell response is selected from the group consisting of: a Th1 response and a Th2 response.

112. The method of claim 111, wherein:
the Th1 or Th2 response is inhibited through induction of an opposing Th2 or Th1 response.

113. The method of claim 109, wherein:
the step of contacting comprises contacting the antigen-exposed pAPC with T cells in the presence of one or more Th1 stimulating cytokines, so that a Th2 response is inhibited.

114. The method of claim 109, wherein:
the step of contacting comprises contacting the antigen-exposed pAPC with T cells in the presence of one or more Th1 stimulating cytokines selected from the group consisting of selected from the group consisting of IL-12, IL-2, IL-18, IL-1β, fragments of IL-1β, IFNα, and IFNγ.

115. The method of claim 109, wherein:
the step of contacting comprises contacting the antigen-exposed pAPC with T cells in the presence of a Th1 inducing agent, so that the expression of or more Th1 cytokines is induced and a Th2 response is inhibited in the T cells.

116. The method of claim 109, wherein:
the step of contacting comprises contacting the antigen-exposed pAPC with T cells in the presence of a Th1 inducing agent selected from the group consisting of selected from the group consisting of LPS, CD40, CD40 ligand, BCGs, oligonucleotides containing CpG motifs, TNFα, and microbial extracts, so that the expression of or more Th1 cytokines is induced and a Th2 response is inhibited in the T cells.

117. The method of claim 116, wherein:
the microbial extracts are selected from the group consisting of any Staphylococcus aureus preparation, heat killed Listeria, and modified cholera toxin.

118. The method of claim 109, wherein:
the step of contacting comprises contacting the mature pAPC displaying antigen with T cells in the presence of one or more Th2 stimulating cytokines

119. The method of claim 109, wherein:
the step of contacting comprises contacting the mature pAPC displaying antigen with T cells in the presence of one or more cytokines selected from the group consisting of IL-4, so that Th1 response is inhibited.

120. The method of claim 109, wherein:
the step of contacting comprises contacting the mature pAPC displaying antigen with T cells in the presence of one or more Th2 inducing agents.

121. The method of claim 109, wherein:
the step of contacting comprises contacting the mature pAPC displaying antigen with T cells in the presence of one or more agents selected to induce expression of IL-4 in the responding T cells.

122. The method of claim 109, wherein:
the pAPC are selected from the group consisting of dendritic cells, B cells, and macrophages.

123. The method of claim 109, wherein:
the pAPC are dendritic cells.

124. The method of claim 123, wherein:
the step of isolating comprises isolating immature dendritic cells from an individual; and
maturing the immature cells in vitro by exposure to one or more cytokines selected from the group consisting of: GM-CSF, IL-3, and IL-4.

125. The method of claim 123, wherein:
the step of maturing is performed concurrently with the step of exposing to antigen.

126. The method of claim 109, wherein:
the step of exposing the isolated cells to an antigen comprises exposing the cells to a crude antigen preparation.

127. The method of claim 109, wherein:
the step of exposing the isolated cells to an antigen comprises exposing the cells to substantially pure antigen.

128. The method of claim 109, wherein:
the step of exposing the isolated cells to antigen comprises exposing the cells to a gene encoding the antigen, so that the gene becomes expressed within the cells.

129. The method of claim 125, wherein:
the step of exposing further comprises exposing the cells to a factor selected from the group consisting of cytokines and inducing agents.

130. The method of claim 129, wherein:
the factor is a polypeptide and the step of exposing comprises contacting the cells with a gene encoding the factor.

131. The method of claim 130, wherein:
the antigen is a polypeptide and the step of exposing comprises contacting the cells with a gene encoding the antigen.

132. The method of claim 131, wherein:
the gene encoding the antigen and the gene encoding the factor are coordinately regulated.

133. The method of claim 130, wherein:
the gene encoding the antigen and the gene encoding the factor are provided on the same nucleic acid molecule.

134. The method of claim 132, wherein:
the gene encoding the antigen and the gene encoding the factor are linked to one another so that a fusion protein is encoded.

135. The method of claim 132, wherein:
the gene encoding the antigen and the gene encoding the factor are provided on separate nucleic acid molecules.

136. The method of claim 109 wherein:
the antigen is provided in association with a targeting agent.

137. The method of claim 129, wherein:
one or both of the antigen and factor is provided in association with a targeting agent.

138. The method of claim 136 or claim 137, wherein:
the association with the targeting agent occurs by means of an interaction selected from the group consisting of covalent bonds, hydrogen bonds, van der Waals interactions, hydrophobic interactions, and combinations thereof.

139. The method of claim 136 or claim 137, wherein:
the targeting agent is selected from the group consisting of mannose receptor ligand and the Fc receptor ligand.

140. The method of claim 136 or claim 137, wherein:
the targeting agent comprises complement receptor ligand.

141. The method of claim 136 or claim 137, wherein:
the targeting agent comprises DEC205.

142. The method of claim 136 or claim 137, wherein:
the targeting agent is capable of targeting to particular vesicles within pAPCs.

143. The method of claim 136 or claim 137, wherein:
the targeting agent comprises at least the Fc portion of an Ig molecule.

144. The method of claim 143, wherein:
the targeting agent comprises at least the Fc portion of an IgG molecule.

145. The method of claim 109, wherein:
the step of exposing comprises providing the antigen in an encapsulation device.

146. The method of claim 129, wherein:
one or both of the antigen and factor is encapsulated.

147. The method of claim 129, wherein:
the antigen and factor are provided together as a single composition.

148. The method of claim 147, wherein:
the antigen and factor are provided encapsulated together in a single encapsulation device.

149. The method of claim 145, 146, or claim 148, wherein:
the encapsulation device is associated with a targeting agent.

150. The method of claim 149, wherein:
the targeting agent is selected from the group consisting of mannose receptor ligand and the Fc receptor ligand.

151. The method of claim 149, wherein:
the targeting agent comprises complement receptor ligand.

152. The method of claim 149, wherein:
the targeting agent comprises DEC205.

153. The method of claim 149, wherein:
the targeting agent is capable of targeting to intracellular vesicles within pAPCs.

154. The method of claim 149, wherein:
the targeting agent comprises at least the Fc portion of an Ig molecule.

155. The method of claim 149, wherein:
the targeting agent comprises at least the Fc portion of an IgG molecule.

156. The method of claim 129, wherein:
the step of exposing comprises providing antigen and factor that are associated with one another by means of an interaction selected from the group consisting of: covalent bonds, hydrogen bonds, van der Waals interactions, hydrophobic interactions, and combinations thereof.

157. The method of claim 109, wherein:
the step of exposing the antigen comprises exposing the cells to a modified antigen.

158. The method of claim 149, wherein:
the antigen comprises an autoantigen; and
the pre-determined set of cytokines comprises Th2 cytokines.

159. The method of claim 149, wherein:
the pre-determined set of cytokines comprises IL-4.

160. A method of treating allergy, the method comprising steps of:
identifying an individual who is allergic to an antigen;
providing a composition of pAPC displaying the antigen; and
contacting the composition with T cells of the individual under conditions that inhibit a Th2 response to the antigen.

161. The method of claim 160, wherein:
the mature pAPC are selected for their expression of Th1 cytokines.

162. The method of claim 160, wherein:
the pAPC are selected from the group consisting of dendritic cells, B cells, and macrophages.

163. The method of claim 161, wherein:
the pAPC are dendritic cells.

164. The method of claim 160, wherein:
the step of providing comprises:
isolating from an individual one or more cells selected from the group consisting of mature pAPC, immature pAPC, and precursors to PAPC; and
exposing the isolated cells to the antigen.

165. The method of claim 164, wherein:
the step of exposing the isolated cells to the antigen further comprises exposing the isolated cells to a factor selected from the group consisting of cytokines and inducing agents.

166. The method of claim 165, wherein:
the factor comprises an inducing agent that induces expression of one or more Th1 stimulating cytokines in the pAPC.

167. The method of claim 165 wherein:
the antigen and factor are provided together as part of a single composition.

168. The method of claim 165, wherein:
one or both of the antigen and factor is associated with a targeting agent.

169. The method of claim 164, wherein:
the antigen is associated with a targeting agent.

170. The method of claim 167, wherein:
the antigen and factor are encapsulated together in an encapsulation device.

171. The method of claim 164, wherein
the antigen is encapsulated.

172. The method of claim 165, wherein:
one or both of the antigen and factor is encapsulated.

173. The method of claim 165, wherein:
the antigen and factor are both encapsulated.

174. The method of claim 173, wherein:
the encapsulation device is associated with a targeting agent.

175. The method of claim 164, wherein:
the step of exposing the isolated cells to antigen comprises exposing the cells to a crude preparation of antigen.

176. The method of claim 164, wherein:
the step of exposing the isolated cells to an antigen comprises exposing the cells substantially pure antigen.

177. The method of claim 164, wherein:
the antigen is a polypeptide antigen; and
the step of exposing the isolated cells to antigen comprises exposing the cells to a gene encoding the antigen, so that the gene becomes expressed within the cells.

178. The method of claim 164, wherein:
the factor is a polypeptide and the step of exposing comprises exposing the cells to a gene encoding the factor.

179. The method of claim 178, wherein:
the antigen is a polypeptide antigen; and
the step of exposing the isolated cells to antigen comprises exposing the cells to a gene encoding the antigen, so that the gene becomes expressed within the cells.

180. The method of claim 179, wherein:
the antigen gene and the factor gene are coordinately regulated.

181. The method of claim 179, wherein:
the antigen gene and the factor gene are provided on the same nucleic acid molecule.

182. The method of claim 181, wherein:
the antigen gene and the factor gene are linked to one another so that a single fusion protein is encoded.

183. The method of claim 179, wherein:
the antigen gene and the factor gene are provided on separate nucleic acid molecules.

184. The method of any one of claims 168, 169, or 174, wherein:
the association with the targeting agent occurs through an interaction selected from the group consisting of covalent bonds, hydrogen bonds, van der Waals interactions, hydrophobic interactions, and combinations thereof.

185. The method of any one of claims 168, 169, or 174, wherein:
the targeting agent is selected from the group consisting of mannose receptor ligand and the Fc receptor ligand.

186. The method of any one of claims 168, 169, or 174, wherein:
the targeting agent comprises complement receptor ligand.

187. The method of any one of claims 168, 169, or 174, wherein:
the targeting agent comprises DEC205.

188. The method of any one of claims 168, 169, or 174, wherein:
the targeting agent is capable of targeting to intracellular vesicles within pAPCs.

189. The method of any one of claims 168, 169, or 174, wherein:
the targeting agent comprises at least the Fc portion of an Ig molecule.

190. The method of any one of claims 168, 169, or 174, wherein:
the targeting agent comprises at least the Fc portion of an IgG molecule.

191. The method of claim 175, wherein:
the step of exposing comprises providing antigen and factor that are associated with one another by means of an interaction selected from the group consisting of: covalent bonds, hydrogen bonds, van der Waals interactions, hydrophobic interactions, and combinations thereof.

192. The method of claim 164, wherein:
the step of exposing the antigen comprises exposing the cells to a modified antigen.

193. The method of claim 192, wherein:
the modified antigen is substantially identical to a naturally-occurring antigen that contains at least one IgE binding site except that the modified antigen lacks at least one of the IgE binding sites.

194. A method of treating an autoimmune disorder, the method comprising steps of:
identifying an individual who is susceptible to or has mounted an undesirable immune response against an antigen;
providing a composition of pAPC displaying the antigen; and
contacting the composition with T cells of the individual under conditions that inhibit a Th1 response to the antigen.

195. The method of claim 194, wherein:
the step of identifying comprises identifying an individual who has previously mounted a Th1 response to the antigen.

196. The method of claim 194, wherein:
the pAPC are selected for their expression of Th2 stimulating cytokines.

197. The method of claim 194, wherein:
the pAPC are selected from the group consisting of dendritic cells, B cells, and macrophages.

198. The method of claim 194, wherein:
the pAPC are B cells.

199. The method of claim 194, wherein:
the step of providing comprises:
isolating from an individual one or more cells selected from the group consisting of mature pAPC, immature pAPC, and precursors to pAPC; and
exposing the isolated cells to the antigen.

200. The method of claim 199, wherein:
the step of exposing the isolated cells to the antigen further comprises exposing the isolated cells to a factor selected from the group consisting of cytokines and inducing agents.

201. The method of claim 200, wherein: the factor comprises an inducing agent that induces expression of one or more Th2 cytokines.

202. The method of claim 200, wherein:
the antigen and factor are provided together as part of a single composition.

203. The method of claim 200, wherein:
one or both of the antigen and factor is associated with a targeting agent.

204. The method of claim 199, wherein:
the antigen is associated with a targeting agent.

205. The method of claim 203, wherein:
the antigen and factor are encapsulated together in an encapsulation device.

206. The method of claim 199, wherein
the antigen is encapsulated.

207. The method of claim 200, wherein:
the antigen and factor are both encapsulated.

208. The method of claim 205, 206, or 207 wherein:
the encapsulation device is associated with a targeting agent.

209. The method of claim 200, wherein:
the step of exposing the isolated cells to antigen comprises exposing the cells to a crude preparation of antigen.

210. The method of claim 200, wherein:
the step of exposing the isolated cells to an antigen comprises exposing the cells substantially pure antigen.

211. The method of claim 199, wherein:
the antigen is a polypeptide antigen; and
the step of exposing the isolated cells to antigen comprises exposing the cells to a gene encoding the antigen, so that the gene becomes expressed within the cells.

212. The method of claim 200, wherein:
the factor is a polypeptide and the step of exposing comprises exposing the cells to a gene encoding the factor.

213. The method of claim 212, wherein:
the antigen is a polypeptide antigen; and
the step of exposing the isolated cells to antigen comprises exposing the cells to a gene encoding the antigen, so that the gene becomes expressed within the cells.

214. The method of claim 213, wherein:
the antigen gene and the factor gene are coordinately regulated.

215. The method of claim 213, wherein:
the antigen gene and the factor gene are provided on the same nucleic acid molecule.

216. The method of claim 215, wherein:
the antigen gene and the factor gene are linked to one another so that a single fusion protein is encoded.

217. The method of claim 213, wherein:
the antigen gene and the factor gene are provided on separate nucleic acid molecules.

218. The method of any one of claims 203, 204, or 208, wherein:
the association with the targeting agent occurs through an interaction selected from the group consisting of covalent bonds, hydrogen bonds, van der Waals interactions, hydrophobic interactions, and combinations thereof.

219. The method of any one of claims 203, 204, or 208, wherein:
the targeting agent is selected from the group consisting of mannose receptor ligand and the Fc receptor ligand.

220. The method of any one of claims 203, 204, or 208, wherein:
the targeting agent comprises complement receptor ligand.

221. The method of any one of claims 203, 204, or 208, wherein:
the targeting agent is capable of targeting to intracellular vesicles within pAPCs.

222. The method of any one of claims 203, 204, or 208, wherein:
the targeting agent comprises at least the Fc portion of an Ig molecule.

223. The method of any one of claims 203, 204, or 208, wherein:
the targeting agent comprises at least the Fc portion of an IgG molecule.

224. The method of claim 200, wherein:
the step of exposing comprises providing antigen and factor that are associated with one another by means of an interaction selected from the group consisting of covalent bonds, van der Waals interactions, hydrophobic interactions, and combinations thereof.

225. The method of claim 199, wherein:
the step of exposing the antigen comprises exposing the cells to a modified antigen.

226. The method of claim 225, wherein:
the modified antigen is substantially identical to a naturally-occurring antigen that contains at least one IgE binding site except that the modified antigen lacks at least one of the IgE binding sites.

227. A composition for modulating an immune system response to an antigen in an individual comprising:
an antigen; and
at least one factor selected from the group consisting of cytokines and inducing agents.

228. The composition of claim 227, wherein:
the factor comprises a Th1 stimulating cytokine.

229. The composition of claim 227, wherein:
the factor is selected from the group consisting of IL-12, IL-2, IL-18, IL-1β, fragments of IL-1β, IFNα, and IFNγ.

230. The composition of claim 227, wherein:
the factor comprises a Th2 stimulating cytokine.

231. The composition of claim 227, wherein:
the factor comprises IL-4.

232. The composition of claim 227, wherein:
the factor comprises a Th1 inducing agent.

233. The composition of claim 227, wherein:
the factor is selected from the group consisting of LPS, CD40, CD40 ligand, BCGs, oligonucleotides containing CpG motifs, TNFα, and microbial extracts.

234. The composition of claim 233, wherein:
the microbial extracts are selected from the group consisting of any Staphylococcus aureus preparation, heat killed Listeria, and modified cholera toxin.

235. The composition of claim 227, wherein:
the factor comprises a Th2 inducing agent.

236. The composition of claim 227, wherein:
the factor comprises an agent that induces IL-4 expression.

237. The composition of claim 227, wherein:
the antigen comprises a crude antigen preparation.

238. The composition of claim 227, wherein:
the antigen comprises a substantially pure antigen

239. The composition of claim 227, further comprising:
an encapsulation device surrounding the antigen and factor.

240. The composition of claim 227 or claim 228, further comprising:
a targeting agent.

241. The composition of claim 240, wherein:
the targeting agent is associated with the composition through a covalent or a non-covalent interaction.

242. The composition of claim 240, wherein:
the targeting agent is selected from the group consisting of mannose receptor ligand and the Fc receptor ligand.

243. The composition of claim 239, wherein:
the targeting agent comprises complement receptor ligand.

244. The composition of claim 239, wherein:
the targeting agent comprises DEC205.

245. The composition of claim 239, wherein:
the targeting agent is capable of targeting to intracellular vesicles within pAPCs.

246. The composition of claim 239, wherein:
the targeting agent comprises at least the Fc portion of an Ig molecule.

247. The composition of claim 239, wherein:
the targeting agent comprises at least the Fc portion of an IgG molecule.

248. The composition of claim 227, wherein:
the antigen and factor are covalently linked to one another.

249. The composition of claim 227, wherein:
the antigen and factor that are associated with one another by means of an interaction selected from the group consisting of: hydrogen bonds, van der Waals interaction, hydrophobic interaction, and combinations thereof.

250. The composition of claim 227, wherein:
the antigen comprises a modified antigen.

251. The composition of claim 227, which composition is formulated for oral administration.

252. The composition of claim 227, which composition is formulated for inhalation.

253. The composition of claim 227, which composition is formulated for injection.

254. A composition for modulating an immune system response to an antigen in an individual comprising:
one or more pAPC displaying an antigen and expressing a predetermined collection of cytokines, selected from the group consisting of Th1 cytokines and Th2 cytokines; and
at least one factor selected from the group consisting of cytokines and inducing agents.

255. The composition of claim 254, wherein:
the pAPC are selected form the group consisting of dendritic cells, B cells, and macrophages.

256. The composition of claim 255, wherein:
the pAPC are dendritic cells.

257. The composition of claim 255, wherein:
the dendritic cells are prepared by a process comprising steps of:
isolating immature dendritic cells from an individual; and
maturing the isolated cells in vitro by exposure to one or more cytokines selected from the group consisting of: GM-CSF, IL-3, and IL-4.

258. The composition of 257, wherein:
the maturing is performed in the presence of the antigen.

259. The composition of claim 254, wherein:
the factor comprises a Th1 stimulating cytokine.

260. The composition of claim 254, wherein:
the factor is selected from the group consisting of IL-12, IL-2, IL-18, IL-1β, fragments of IL-1β, IFNα, and IFNγ

261. The composition of claim 254, wherein:
the factor comprises a Th1 inducing agent.

262. The composition of claim 254, wherein:
the factor is selected from the group consisting of LPS, CD40, CD40 ligand, BCGs, oligonucleotides containing CpG motifs, TNFα, and microbial extracts.

263. The method of claim 262, wherein:
the microbial extracts are selected from the group consisting of any Staphylococcus aureus preparation, heat killed Listeria, and modified cholera toxin.

264. The composition of claim 254, wherein:
the factor comprises a Th2 stimulating cytokine.

265. The composition of claim 254, wherein:
the factor comprises IL-4.

266. The composition of claim 254, wherein:
the factor comprises a Th2 inducing agent.

267. The composition of claim 254, wherein:
the factor comprises an agent that induces IL-4 expression.

268. The composition of claim 254, wherein:
the factor comprises an agent that inhibits IL-12 expression.

269. The composition of claim 258, wherein:
the antigen comprises a crude antigen preparation.

270. The composition of claim 254, wherein:
the antigen comprises a substantially pure antigen.

271. The composition of claim 254, wherein:
the antigen comprises a modified antigen.

272. A composition comprising:
a gene encoding an antigen; and
a gene encoding at least one factor selected from the group consisting of cytokines and inducing agents.

273. The composition of claim 272, wherein:
the antigen gene and the factor gene are coordinately regulated.

274. The composition of claim 272, wherein:
the antigen gene and the factor gene are on the same nucleic acid molecule.

275. The composition of claim 272, wherein:
the antigen gene and the factor gene are linked together so that a single polypeptide is encoded.

276. The composition of claim 272, wherein:
the antigen gene and the factor gene are provided on separate nucleic acid molecules.

277. The composition of claim 272, further comprising an encapsulation device surrounding the genes.

278. The composition of claim 272 or claim 277, further comprising:
a targeting agent selected for its ability to localize the composition in the vicinity of pAPC.

279. The composition of claim 272, which composition is formulated for oral administration.

280. The composition of claim 272, which composition is formulated for inhalation.

281. The composition of claim 272, which composition is formulated for injection.
